SEQUENCE LISTING (11 sequences)

SEQ ID NO: 1 - protein, 609 aa, Homo sapiens [sequence omitted]

SEQ ID NO: 2 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 474 aa, Homo sapiens [sequence omitted]

SEQ ID NO: 4 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - protein, 1006 aa, Homo sapiens
MKILILGIFLFLCSTPAWAKEKHYYIGIIETTWDYASDHGEKKLISVDTEHSNIYLQNGPDRIGRLYKKALYLQYTDETFRTTIEKPVWLGFLGPIIKAETGDKVYVHLKNLASRPYTFHSHGITYYKEHEGAIYPDNTTDFQRADDKVYPGEQYTYMLLATEEQSPGEGDGNCVTRIYHSHIDAPKDIASGLIGPLIICKKDSLDKEKEKHIDREFVVMFSVVDENFSWYLEDNIKTYCSEPEKVDKDNEDFQESNRMYSVNGYTFGSLPGLSMCAEDRVKWYLFGMGNEVDVHAAFFHGQALTNKNYRIDTINLFPATLFDAYMVAQNPGEWMLSCQNLNHLKAGLQAFFQVQECNKSSSKDNIRGKHVRHYYIAAEEIIWNYAPSGIDIFTKENLTAPGSDSAVFFEQGTTRIGGSYKKLVYREYTDASFTNRKERGPEEEHLGILGPVIWAEVGDTIRVTFHNKGAYPLSIEPIGVRFNKNNEGTYYSPNYNPQSRSVPPSASHVAPTETFTYEWTVPKEVGPTNADPVCLAKMYYSAVDPTKDIFTGLIGPMKICKKGSLHANGRQKDVDKEFYLFPTVFDENESLLLEDNIRMFTTAPDQVDKEDEDFQESNKMHSMNGFMYGNQPGLTMCKGDSVVWYLFSAGNEADVHGIYFSGNTYLWRGERRDTANLFPQTSLTLHMWPDTEGTFNVECLTTDHYTGGMKQKYTVNQCRRQSEDSTFYLGERTYYIAAVEVEWDYSPQREWEKELHHLQEQNVSNAFLDKGEFYIGSKYKKVVYRQYTDSTFRVPVERKAEEEHLGILGPQLHADVGDKVKIIFKNMATRPYSIHAHGVQTESSTVTPTLPGETLTYVWKIPERSGAGTEDSACIPWAYYSTVDQVKDLYSGLIGPLIVCRRPYLKVFNPRRKLEFALLFLVFDENESWYLDDNIKTYSDHPEKVNKDDEEFIESNKMHAINGRMFGNLQGLTMHVGDEVNWYLMGMGNEIDLHTVHFHGHSFQYK

SEQ ID NO: 6 - protein, 1246 aa, Homo sapiens
MREWVLLMSVLLCGLAGPTHLFQPSLVLDMAKVLLDNYCFPENLLGMQEAIQQAIKSHEILSISDPQTLASVLTAGVQSSLNDPRLVISYEPSTPEPPPQVPALTSLSEEELLAWLQRGLRHEVLEGNVGYLRVDSVPGQEVLSMMGEFLVAHVWGNLMGTSALVLDLRHCTGGQVSGIPYIISYLHPGNTILHVDTIYNRPSNTTTEIWTLPQVLGERYGADKDVVVLTSSQTRGVAEDIAHILKQMRRAIVVGERTGGGALDLRKLRIGESDFFFTVPVSRSLGPLGGGSQTWEGSGVLPCVGTPAEQALEKALAILTLRSALPGVVHCLQEVLKDYYTLVDRVPTLLQHLASMDFSTVVSEEDLVTKLNAGLQAASEDPRLLVRAIGPTETPSWPAPDAAAEDSPGVAPELPEDEAIRQALVDSVFQVSVLPGNVGYLRFDSFADASVLGVLAPYVLRQVWEPLQDTEHLIMDLRHNPGGPSSAVPLLLSYFQGPEAGPVHLFTTYDRRTNITQEHFSHMELPGPRYSTQRGVYLLTSHRTATAAEEFAFLMQSLGWATLVGEITAGNLLHTRTVPLLDTPEGSLALTVPVLTFIDNHGEAWLGGGVVPDAIVLAEEALDKAQEVLEFHQSLGALVEGTGHLLEAHYARPEVVGQTSALLRAKLAQGAYRTAVDLESLASQLTADLQEVSGDHRLLVFHSPGELVVEEAPPPPPAVPSPEELTYLIEALFKTEVLPGQLGYLRFDAMAELETVKAVGPQLVRLVWQQLVDTAALVIDLRYNPGSYSTAIPLLCSYFFEAEPRQHLYSVFDRATSKVTEVWTLPQVAGQRYGSHKDLYILMSHTSGSAAEAFAHTMQDLQRATVIGEPTAGGALSVGIYQVGSSPLYASMPTQMAMSATTGKAWDLAGVEPDITVPMSEALSIAQDIVALRAKVPTVLQTAGKLVADNYASAELGAKMATKLSGLQSRYSRVTSEVALAEILGADLQMLSGDPHLKAAHIPENAKDRIPGIVPMQIPSPEVFEELIKFSFHTNVLEDNIGYLRFDMFGDGELLTQVSRLLVEHIWKKIMHTDAMIIDMRFNIGGPTSSIPILCSYFFDEGPPVLLDKIYSRPDDSVSELWTHAQVVGERYGSKKSMVILTSSVTAGTAEEFTYIMKRLGRALVIGEVTSGGCQPPQTYHVDDTNLYLTIPTARSVGASDGSSWEGVGVTPHVVVPAEEALARAKEMLQHNQLRVKRSPGLQDHL

SEQ ID NO: 7 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 8 - protein, 142 aa, Homo sapiens
MVLSPADKTNVKAAWGKVGAHAGEYGAEALERMFLSFPTTKTYFPHFDLSHGSAQVKGHGKKVADALTNAVAHVDDMPNALSALSDLHAHKLRVDPVNFKLLSHCLLVTLAAHLPAEFTPAVHASLDKFLASVSTVLTSKYR

SEQ ID NO: 9 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 10 - protein, 16 aa, sequence artificielle
  MISC_FEATURE (9)..(9): tyrosine nitree (Tyr-NO2)
CEETFLKKXLYEIARR

SEQ ID NO: 11 - protein, 9 aa, sequence artificielle
  MISC_FEATURE (4)..(4): tyrosine nitree (Tyr-NO2)
LVRXTKKVC

(19) United States
(12) Patent Application Publication
(10) Pub. No.: US 2011/0033868 A1
(43) Pub. Date: Feb. 10, 2011

(54) USE OF A NITRATED PROTEIN OR PEPTIDE SEQUENCE FOR THE IMPLEMENTATION OF A METHOD OF DIAGNOSIS

(75) Inventor: Serge Bottari, Biviers (FR)

Correspondence Address: YOUNG & THOMPSON, 209 Madison Street, Suite 500, Alexandria, VA, 22314, US

(73) Assignee: UNIVERSITE JOSEPH FOURIER, Grenoble Cedex 09 (FR)

(21) Appl. No.: 12/812,498

(22) PCT Filed: Jan. 12, 2009
(86) PCT No.: PCT/FR09/00036

§ 371 Date: Oct. 12, 2010

(30) Foreign Application Priority Data
Jan. 10, 2008 (FR) 0800142

(51) Int. Cl. G01N 33/566; C07K 16/18; C12N 5/16; C07K 1/00

(52) U.S. Cl. 435/7.1; 436/501; 530/388.2; 435/332; 530/409

(57) ABSTRACT

The present invention relates to the use of quantitative assay, in particular in vitro, in a biological sample, of the degree of nitration of tyrosine residues of a particular nitrated protein or physiological peptide sequence, for the implementation of a method of in vitro diagnosis of the state of severity and progressiveness of a chronic or acute pathology associated with nitrating stress.

The present invention relates to the use of a nitrated protein or peptide sequence for the implementation of a method of diagnosis.

The present invention relates more particularly to the use of a nitrated protein or peptide sequence for implementing the diagnosis of the state of severity and progressiveness of associated pathologies, involving or due to nitrating stress.

Oxidative stress is a type of attack of the cellular constituents due to the oxidative Reactive Oxygen Species (ROS) and Reactive Nitrogen Species (RNS). These species are, by definition, free radicals. By association, hydrogen peroxide (H₂O₂) is regarded as an ROS because, in the presence of iron (in ionic form), it is transformed into two hydroxyl radicals (OH.) (Haber-Weiss Reaction).

The production of ROS and RNS is normal for all organisms that live in aerobic conditions and does not in itself constitute a situation of oxidative stress because the cell has at its disposal a complex detoxifying system against ROS comprising enzymes (superoxide dismutase, catalase, glutathione peroxidase etc.) and small molecules (vitamin E, vitamin C, glutathione etc.). Under physiological conditions, the superoxide anion (O₂⁻) is produced essentially by the NADPH oxidases (NOX) and nitrogen monoxide (NO.) by the family of the NO synthases.

Oxidative stress becomes a pathological situation once the defence system is overwhelmed by the ROS and RNS. This may be due for example to:
the introduction of free radicals or of oxygen-containing reactive species into the cell (chemical pollutants entering the body via the respiratory system, the alimentary canal or the mucosae)
overproduction of ROS and RNS induced by hypoxia or processes of the ischaemia-reperfusion type, which are the cause of some transplant rejections or the presence of certain pro-oxidative chemical compounds.
dysfunction of the mitochondrial respiratory chain, for example following hypoxia, hypoglycaemia or xenobiotics acting on certain of its complexes.
activation of xanthine oxidase
increase in expression or activity of NOS II for example following the triggering of an inflammatory reaction.
a defect in the defence system, for example a mutation or xenobiotics inactivating one of the enzymes of the defence system or a deficiency of one of the antioxidant vitamins (vitamins C and E).
the introduction of highly reactive molecules into the cell or into an organ, for example nanoparticles (very small and with highly developed specific surface). If these nanoparticles are numerous, the macrophages are no longer able to deal with them and may release their oxidants in the organism, causing an exacerbated inflammatory reaction.

The functional consequences of oxidative stress vary widely depending on its intensity. Recent works indicate that the ratio of superoxide ions to nitrogen monoxide (O₂⁻/NO) is decisive. In fact, while this ratio≦1, the O₂⁻ reacts preferentially with NO, permitting the appearance of nitrogen-containing radical species (RNS), nitrosonium (NO⁺) and peroxynitrite (ONOO⁻), which induce post-translational modifications. These RNS induce respectively nitrosation (R-Cys-SH→R-Cys-SNO) and nitration (R-Tyr→R-Tyr-NO₂) of the proteins. In contrast to the modifications induced by the ROS species, those induced by the RNS species are reversible.

When the ratio O₂⁻/NO>1, the O₂.⁻ ions, then the OH.⁻ “free” radicals induce irreversible oxidation of proteins, lipids and nucleic acids, which can be measured by means of numerous plasma markers.

These modifications reflect, moreover, one of the mechanisms of cellular defence against oxidative stress: the production of NO. It can therefore be postulated that once the cell's capacity to “absorb” the NO⁺ and the ONOO⁻ produced, the latter being diffusible through the plasma membrane, they will be able respectively to nitrosate and nitrate extracellular proteins, including the plasma proteins.

Oxidative and nitrating stress are factors in inflammation and mutagenesis. They are also regarded as one of the principal causes of cancer and are thought to play a role in neurodegenerative diseases (Parkinson's, Alzheimer's, multiple sclerosis, ALS), as well as in several more common pathologies such as type 1 and 2 diabetes, cardiovascular diseases, cerebrovascular accidents, rheumatoid arthritis or cataract.

To date, there is no method for determining nitrating stress by assay of a circulating marker specifically and quantitatively.

Khan et al. (Khan et al. Biochem J, 1998, 330, 795-801) disclose a method for assaying the nitrated proteins on the basis of tyrosine residues (ELISA assay) using antibodies that recognize the nitrated tyrosines of all the proteins.

It is clearly stipulated that the method described by Khan et al. provides a qualitative assay of the nitrated proteins, but this does not give an accurate and reliable measure of the degree of nitration of the circulating proteins. In fact, Khan et al. disclose the use of antibodies capable of recognizing all the nitrated proteins and not specific proteins.

Methods of detecting the quantity of NO associated with proteins are described in the prior art.

In particular, application WO/1998/029452 describes antibodies that recognize the nitrated tyrosines, in particular the nitrated tyrosines of the NO transporter proteins, for their use as therapeutic agents for treating pathologies associated with the nitrosylation and nitration of proteins.

Furthermore, US application 2005/244905 describes a method of diagnosing the risk of coronary diseases in patients by detecting the degree of nitration of fibrinogen based on tyrosine. This method uses a pair of antibodies, permitting said detection of nitrated fibrinogen: a capturing antibody that detects all the nitrated tyrosines, and an antibody that specifically detects fibrinogen, whether or not containing nitrogen.

Other examples of methods of detecting nitrated proteins on the basis of tyrosines are also described in the prior art.

For example, international application WO 03/076946 describes the use of an immunologic “partner” capable of detecting a specific epitope containing an aromatic nitrated amino acid. More particularly, WO 03/076946 describes the use of a specific antibody recognizing an epitope of type II collagen in which a tyrosine is nitrated. The antibody described in this document is used for diagnostic purposes for evaluating the degree of oxidation of the proteins, and more particularly of type 2.2 collagen within the scope of pathologies associated with nitrating stress. However, at no point in WO 03/076946 is there any description that it is possible to detect the state of progression or the progressiveness of a pathology associated with nitrating stress.

Although these documents disclose antibodies that recognize nitrated proteins, nothing is said concerning the production of specific antibodies directed against nitrated residues, particularly the tyrosine residues Y¹³⁸ and Y⁴¹¹ of albumin, or any other residue with a defined position in circulating proteins.

The nitration of albumin has been described in the prior art. In particular, Jiao et al. (Analytical Biochem, 2001, 293, 43-52) disclose the nitrated residues of albumin. Jiao et al. describe the nitration of the Y¹³⁸ and Y⁴¹¹ residues of albumin, after in vitro nitration of albumin by peroxynitrite, and identification of the sites of nitration by mass spectrometry.

Malan, P. G., et al. (1970) Biochemistry 9(16), 3205-3214 and Sokolovsky, et al. (1966) Biochemistry 5(11), 3582-3589 for their part describe methods of in vitro nitration of proteins using tetranitromethane as nitrating agent.

One aspect of the invention is to provide a method of diagnosing the state of severity and progressiveness of pathologies due to or associated with, or causing, nitrating stress.

Another aspect of the invention is to provide a method of in vitro quantitative assay in vitro of the degree of nitration of physiological proteins.

Another aspect of the invention is to permit determination of the position of the nitrated tyrosines in physiological proteins, under the influence of nitrating stress.

The present invention relates to the use of quantitative assay, in particular in vitro, in a biological sample, of the degree of nitration of tyrosine residues of a particular nitrated protein or physiological peptide sequence, for the implementation of a method of in vitro diagnosis of the state of severity and progressiveness of a chronic or acute pathology associated with nitrating stress.

The present invention is based on the unexpected finding of the existence of a correlation between the value of the degree of physiological nitration of proteins and the state of severity and progressiveness of a pathology associated with nitrating stress.

In the invention, “quantitative assay of the degree of nitration” means the action that consists of accurately determining the quantity of particular nitrated proteins. The degree of nitration of the particular nitrated protein is quantified by establishing the ratio of the quantity of particular nitrated protein to the total quantity of said particular protein, nitrated and non-nitrated. This value is therefore between 0 and 1.

In the invention, “particular protein or physiological peptide sequence” denotes any protein, peptide, fragment of proteins or of peptides, or any known sequence of at least 2 amino acids that is synthesized within a living organism, generally without human external intervention. It is also understood that the terms “particular protein or physiological peptide sequence” correspond to “a particular physiological protein” or to “a particular physiological peptide sequence”. According to one embodiment of the invention, the particular proteins in question are circulating proteins, occurring in the blood, the cerebrospinal fluid or the urine. Other proteins are not excluded from the invention.

Protein also means, hereinafter, any sequence of amino acids having antigenic properties, and therefore capable of permitting reaction of the immune system and thus of generating antibodies. These antibodies are then in their turn capable of recognizing said protein which permitted their synthesis.

“Nitrated” defines, in the invention, the presence of an —NO₂ group bound covalently to a protein or a peptide sequence. Also, nitrated protein defines a protein in which one or more tyrosines have, on their phenol group, an —NO₂ group in position 3. These —NO₂ groups attach to the proteins, in vivo, following the production of peroxynitrite, which can react with certain particular tyrosine residues of particular proteins.

Chronic pathology means, hereinafter, any disorder of long duration and generally with a poor prognosis and frequently accompanied by complications in the form of associated pathologies, which in their turn can be acute or chronic. Acute pathology means any disorder manifested by symptoms of varying severity ending after a relatively short period either in cure or death.

“The state of severity and progressiveness of a pathology” is defined in the invention as a defined stage, characterized in that it describes a particular state of said pathology, and said state can range from absence of symptoms that are characteristic of said pathology to the most advanced state, i.e. where all the symptoms described so far are cumulative. Characterization of the state of severity and progressiveness of the pathology is based on the clinical and physiopathological knowledge of said pathology.

In the invention, a correlation is defined between degree of nitration and nitrating stress. Nitrating stress defines an overproduction of peroxynitrite (ONOO⁻) relative to the quantity of peroxynitrite produced in an individual declared healthy. This means that the greater the quantity of peroxynitrite in the organism, the more the nitrated species derived from peroxynitrite will be capable of nitrating the proteins. Also, the higher the nitrating stress, the more the proteins will be nitratable.

According to an advantageous embodiment of the invention, the quantitative assay is carried out by means of at least one specific antibody recognizing a physiologically nitrated tyrosine of said particular nitrated protein or physiological peptide sequence.

“Nitrated tyrosine” means, in the invention, the modified tyrosine residue after addition of the —NO₂ group to the aromatic ring of the tyrosine. The NO₂ thus present on the tyrosine residue is preferably in position 3. The terms “tyrosine” and “tyrosine residue” are used uniformly in the invention to denote the amino acid in question.

In the invention, the expression “specific antibody recognizing a nitrated tyrosine” denotes an antibody recognizing a specific, or particular, immunogenic amino acid sequence, in which there is a nitrated tyrosine residue. The recognition is called specific, which means that the immunogenic amino acid sequence in which there is a non-nitrated tyrosine residue is not recognized by said specific antibody, and that an immunogenic amino acid sequence different from the specific, or particular, immunogenic amino acid sequence containing a tyrosine, or a tyrosine residue, even nitrated, will not be recognized by said specific antibody.

According to another advantageous embodiment of the invention, the particular nitrated physiological protein or peptide sequence is preferably a, in particular plasma, circulating protein, in particular selected from the following proteins: albumin, prealbumin, vitamin D binding protein (VDBP), transferrin, ceruloplasmin, retinol binding protein (RBP), insulin, haemoglobin, β actin, band 3 protein of the erythrocyte anion transporter, β chain of erythrocyte spectrin, fibronectin precursor, β chain of fibrinogen and erythrocyte membrane protein band 4.1.

“Circulating protein” is defined in the invention as proteins that occur naturally in blood, plasma and possibly lymph, cerebrospinal fluid, saliva or urine.

Said proteins: albumin, prealbumin, vitamin D binding protein (VDBP), transferrin, ceruloplasmin, retinol binding protein (RBP), insulin, haemoglobin, β actin, anion transporter band 3 protein, β chain of erythrocyte spectrin, fibronectin precursor, β chain of fibrinogen and erythrocyte membrane protein band 4.1, described in the invention are circulating proteins, present in the blood of humans or of animals and that can be purified or produced in vitro by the standard techniques known by a person skilled in the art.

The prealbumin described in the invention is also commonly called transthyretin.

In the invention, β actin may also be called actin β.

Haemoglobin is a protein characterized in that it comprises 4 subunits: two subunits of haemoglobin α and two subunits of haemoglobin β.

It should also be noted that the band 3 protein of the erythrocyte anion transporter corresponds to the CD233 antigen.

The proteins involved in the invention, in their non-nitrated forms, are represented by the following sequences: albumin is represented by the sequence SEQ ID NO: 1, transthyretin is represented by the sequence SEQ ID NO: 2, VDBP by the sequence SEQ ID NO: 3, transferrin by the sequence SEQ ID NO: 4, ceruloplasmin by the sequence SEQ ID NO: 5, RBP by the sequence SEQ ID NO: 6, insulin by the sequence SEQ ID NO: 7, haemoglobin a by the sequence SEQ ID NO: 8, haemoglobin β by the sequence SEQ ID NO: 9, β actin by the sequence SEQ ID NO: 10, the band 3 protein of the erythrocyte anion transporter by the sequence SEQ ID NO: 11, the β chain of erythrocyte spectrin by the sequence SEQ ID NO: 12, the fibronectin precursor by the sequence SEQ ID NO: 13, the β chain of fibrinogen by the sequence SEQ ID NO: 14 and the erythrocyte membrane protein band 4.1 by the sequence SEQ ID NO: 15.

In the invention, the preceding proteins, in their non-nitrated forms represented by the sequences SEQ ID NO 1 to SEQ ID NO 15, are also represented by the variants or isoforms of said sequences, or any protein having a sequence identity of at least 90%, and more particularly 100%, with said protein.

According to another preferred embodiment of the invention, said chronic or acute pathologies associated with nitrating stress belong to the following group: inflammatory and autoimmune diseases, infectious diseases, neurodegenerative diseases, hypoxic and ischaemic diseases, type 1 and 2 diabetes, metabolic disorders, hyper- and hypothyroidism, cardiovascular and respiratory diseases, and cancer.

In a preferred embodiment, the chronic or acute pathologies associated with nitrating stress in the invention correspond to:
neurodegenerative diseases, including among others Parkinson's disease, Alzheimer's disease, amyotrophic lateral sclerosis, multiple sclerosis, periventricular leukomalacia or Creutzfeldt-Jakob disease,
ischaemic diseases, including among others coronary diseases, myocardial infarction, cerebrovascular accidents, shock or preeclampsia and eclampsia,
diseases associated with hypoxia, such as chronic obstructive bronchopathies, asthma, emphysema, nicotinism, fibrosing pneumopathies, sleep apnoea syndrome, antenatal or neonatal hypoxia as well as encephalopathies connected with peripartal asphyxia,
type 1 and 2 diabetes, as well as insulin resistance, neonatal hypoglycaemia, hypoglycaemia occurring in poorly managed or untreated diabetes as well as more generally all complications of diabetes such as, non-limitatively, diabetic retinopathy, nephropathy, neuropathy, arteriopathy and cardiopathy,
cardiovascular diseases including, among others, atherosclerosis, heart failure and decompensation or arterial hypertension and pulmonary artery hypertension, and
complications in transplant patients such as bone marrow, kidney, heart, heart and lung and liver transplants.
inflammatory diseases whether or not of infectious origin as well as autoimmune diseases such as rheumatoid polyarthritis, osteoarthritis, ankylosing spondylitis, scleroderma, lupus erythematosus disseminatus or any other forms of lupus, Sjögren syndrome, Goodpasture syndrome, temporal arteritis, sarcoidosis, multiple sclerosis, autoimmune thrombocytopenic purpura, autoimmune haemolytic anaemia, pemphigus, polymyositis, fibromyalgia etc.

In another preferred embodiment of the invention, the chronic or acute pathologies associated with nitrating stress correspond to the aforementioned pathologies but excluding antenatal or neonatal hypoxia, encephalopathies connected with peripartal asphyxia and neonatal hypoglycaemia.

In other words, an even more preferred embodiment of the invention describes chronic or acute pathologies associated with nitrating stress in the invention corresponding to
neurodegenerative diseases, including among others Parkinson's disease, Alzheimer's disease, amyotrophic lateral sclerosis, multiple sclerosis, periventricular leukomalacia or Creutzfeldt-Jakob disease,
ischaemic diseases, including among others coronary diseases, myocardial infarction, cerebrovascular accidents, shock or preeclampsia and eclampsia,
diseases associated with hypoxia, such as chronic obstructive bronchopathies, asthma, emphysema, nicotinism, fibrosing pneumopathies and sleep apnoea syndrome,
type 1 and 2 diabetes, as well as insulin resistance preceding type 2 diabetes, hypoglycaemia occurring in poorly managed or untreated diabetes as well as more generally all complications of diabetes such as, non-limitatively, diabetic retinopathy, nephropathy, neuropathy, arteriopathy and cardiopathy,
cardiovascular diseases including, among others, atherosclerosis, heart failure and decompensation or arterial hypertension and pulmonary artery hypertension, and
complications in transplant patients such as bone marrow, kidney, heart, heart and lung and liver transplants,
inflammatory diseases whether or not of infectious origin as well as autoimmune diseases such as rheumatoid polyarthritis, osteoarthritis, ankylosing spondylitis, scleroderma, lupus erythematosus disseminatus or any other forms of lupus, Sjögren syndrome, Goodpasture syndrome, temporal arteritis, sarcoidosis, multiple sclerosis, autoimmune thrombocytopenic purpura, autoimmune haemolytic anaemia, pemphigus, polymyositis, fibromyalgia etc.

According to another advantageous embodiment of the invention, said particular nitrated protein or physiological peptide sequence involved in the invention is nitrated albumin.

According to another advantageous embodiment of the invention, the quantitative assay, in particular in vitro, mentioned previously, is carried out by means of a specific antibody specifically recognizing the nitrated Y¹³⁸ tyrosine residue of albumin, in particular a monoclonal antibody.

“Y¹³⁸ tyrosine residue” means, in the invention, the tyrosine in position 138 in human albumin. The invention also relates to the tyrosine in the equivalent position in the albumins of other non-human mammals.

In the invention, the Y¹³⁸ residue of human albumin (the albumin being represented in its non-nitrated form by the sequence SEQ ID NO 1) is contained, in its nitrated form, in the peptide of sequence SEQ ID NO 16.

According to another advantageous embodiment of the invention, the quantitative assay, in particular in vitro, mentioned previously, is carried out by means of a specific antibody specifically recognizing the nitrated Y⁴¹¹ tyrosine residue of albumin, in particular a monoclonal antibody.

“Y⁴¹¹ tyrosine residue” means, in the invention, the tyrosine in position 411 in human albumin. The invention also relates to the tyrosine in the equivalent position in the albumins of other non-human mammals.

In the invention, the Y⁴¹¹ residue of human albumin (the albumin being represented in its non-nitrated forms by the sequence SEQ ID NO 1) is contained in its nitrated forms in the peptide of sequence SEQ ID NO 17.

The invention also relates to an antibody specifically recognizing nitrated albumin on the Y¹³⁸ tyrosine residue, in particular a monoclonal antibody.

According to a preferred embodiment, the invention describes a monoclonal antibody as mentioned above, secreted by the hybridoma deposited according to the Treaty of Budapest at the CNCM (Collection Nationale de Culture de Microorganismes, Institut Pasteur, 25, rue du Docteur Roux, 75724 Paris CEDEX 15, France) on 8 Jan. 2009, under the accession number CNCM I-4111.

Said aforementioned monoclonal antibody is obtained by the immunization of mice by means of the peptide SEQ ID NO 16, according to the procedure described below in the experimental section. Said antibody is also called hereinafter 13H10-3G12-3A6 antibody or 13H10 antibody (clone). This monoclonal antibody is of isotype IgG2b.

The invention also relates to an antibody specifically recognizing nitrated albumin on the Y⁴¹¹ tyrosine residue, in particular a monoclonal antibody.

According to a preferred embodiment, the invention describes a monoclonal antibody mentioned above, secreted by the hybridoma deposited according to the Treaty of Budapest at the CNCM (Collection Nationale de Culture de Microorganismes, Institut Pasteur, 25, rue du Docteur Roux, 75724 Paris CEDEX 15, France) on 8 Jan. 2009, under the accession number CNCM I-4110.

Said aforementioned monoclonal antibody is obtained by the immunization of mice by means of the peptide SEQ ID NO 17, according to the procedure described below in the experimental section. Said antibody is also called hereinafter 2F3-2E2 antibody or 2F3 antibody (clone). This monoclonal antibody is of isotype IgG1.

The antibodies of the invention are both polyclonal and monoclonal antibodies.

The two antibodies of the invention are more particularly monoclonal antibodies. The term antibody in the invention includes all fragments derived from antibodies, in particular the fragments of said monoclonal antibodies having substantially the same antigenic specificity for the particular nitrated protein. These fragments comprise antibody fragments (i.e. Fab, F(ab′)2, CDRs, etc.), polyfunctional antibodies, single-chain antibodies (scFv) etc. The antibodies of the invention can be produced by conventional methods, comprising the immunization of an animal and recovery of the splenic cells so as to produce hybridomas by cellular fusion. The antibodies of the invention can be used advantageously in the form of a mixture of monoclonal antibodies.

The methods of production of the monoclonal antibodies are known by a person skilled in the art. They generally comprise the immunization of a non-human animal with an antigen, followed by recovery of the thymus cells from the animal, which are fused with immortalized cells, generally myeloma cells. The resultant hybridomas produce monoclonal antibodies.

The advantageous antibodies of the invention are prepared by immunization of non-human animals by means of specific peptides of the sequences that include the substantially pure nitrated Y¹³⁸ and Y⁴¹¹ tyrosines of albumin. These peptides have the following sequences:
EETFLKK(Y¹³⁸-NO₂)LYEIARR—comprising Y¹³⁸ tyrosine, and represented by the sequence SEQ ID NO: 16.
LVR(Y⁴¹¹-NO₂)TKKV—comprising Y⁴¹¹ tyrosine, and represented by the sequence SEQ ID NO: 17.

The above peptides are novel.

The invention also has the aim of supplying the hybridoma deposited according to the Treaty of Budapest at the CNCM (Collection Nationale de Culture de Microorganismes, Institut Pasteur, 25, rue du Docteur Roux, 75724 Paris CEDEX 15, France) on 8 Jan. 2009, under the accession number CNCM I-4111.

Moreover, the invention describes the hybridoma deposited according to the Treaty of Budapest at the CNCM (Collection Nationale de Culture de Microorganismes, Institut Pasteur, 25, rue du Docteur Roux, 75724 Paris CEDEX 15, France) on 8 Jan. 2009, under the accession number CNCM I-4110.

These two hybridomas are novel. The methods of obtaining these two hybridomas are described in the examples given below.

The invention also has the aim of supplying a method for in vitro diagnosis of the state of severity and progressiveness of a chronic or acute pathology associated with nitrating stress in a biological sample from an individual, comprising:
quantitative assay, in a biological sample from an individual, of the degree of nitration of a first particular nitrated protein or physiological peptide sequence,
quantification of said degree of nitration of said first particular protein or physiological peptide sequence by comparing the concentration of said first particular nitrated protein or physiological peptide sequence with the total concentration of said first particular protein or physiological peptide sequence, nitrated and non-nitrated, obtained from a biological sample from the same individual,
comparing said degree of nitration of said first particular protein or physiological peptide sequence with the degree of nitration of a second particular protein or physiological peptide sequence obtained from a biological sample from an individual not affected by said chronic or acute pathology,
   said second particular protein or physiological peptide sequence being a variant or an isoform, or having an amino acid sequence identity of at least 90%, and in particular 100%, with the aforesaid first particular protein or physiological peptide sequence,
   said first particular protein or physiological peptide sequence being nitrated on at least one tyrosine residue located in an equivalent position in the aforesaid second particular protein or physiological peptide sequence,
deduction, based on the comparison carried out in the preceding stage, of the degree of nitrating stress of the individual that may correspond to a severity and progressiveness of said chronic or acute pathology.

This method is based on the discovery by the inventors of a method of in vitro nitration of proteins permitting nitration on tyrosine residues equivalent to the residues nitrated physiologically.

The degree of nitration of the particular nitrated protein is quantified by establishing the ratio of the quantity of particular nitrated protein to the total quantity of said particular protein, nitrated and non-nitrated. This value is therefore ideally between 0 and 1.

“First physiological protein” means, hereinafter, any physiological protein corresponding to the physiological protein under investigation, taken from the biological sample from the individual in question, namely the individual being tested.

“Second physiological protein” defines a protein having a sequence identity of at least 90%, and more particularly 100%, with said first particular protein, said second protein being obtained from a biological sample, of the same nature or of a different nature, from an individual other than the individual from whom the first particular protein was obtained. In particular the individual from whom the second particular nitrated protein is obtained corresponds to an individual who is not affected by said pathology associated with nitrating stress.

“Variant” or “isoform” of the particular protein defines any protein, peptide or polypeptide encoded by one and the same wild-type or mutant gene, the amino acid sequences of which are close to that of the particular protein and have a sequence identity of at least 90%, and more particularly 100%, with the protein in question and which generally has the same biological function as said protein in question. Therefore variants derived from gene mutations and displaying gains or losses of function are not excluded. These variants or isoforms can be, for example, the result of an alternative splicing of one and the same gene or of the expression of several homologous genes the sequences of which have diverged.

The first physiological protein and the second physiological protein can be the same proteins, except for the nitration or non-nitration of one or more tyrosine residues.

“Equivalent position” of tyrosine between the first and the second protein means, in the invention, a position in the amino acid sequence of the first particular protein which corresponds to the position of a tyrosine in the second particular protein such that, if the two proteins are aligned according to standard sequence alignment procedures (Needleman-Wunsch algorithm, Smith-Waterman algorithm etc.), these two residues occur in the same position. More explicitly, this means that even if the two proteins are not of equal size, following alignment, the two residues will be found in identical positions relative to the surrounding sequences.

The method also described in the invention therefore consists of an assay of the degree of physiological or pathological nitration of a first particular physiological protein of the invention, from a biological sample from an individual. The advantageous biological sample in the invention can be blood or a blood derivative (plasma, serum, corpuscles, platelets). Biological samples such as urine, cerebrospinal fluid, tissues, tumours, cells, blood smears, etc. can also be involved in the invention. The assay therefore consists of evaluating the nitration of the nitrated physiological proteins. A final value of the degree of physiological nitration of said particular protein is therefore established.

The previously determined degree of nitration is compared with the degree of nitration of the second particular protein obtained from a biological sample from an individual regarded as not affected by one of the pathologies of the invention.

To date, the normal degrees of nitration of the physiological proteins are not known quantitatively. They are generally estimated as being low (<5%).

The degrees of nitration of the first and of the second particular physiological protein are then compared. When the degree of nitration of the first nitrated protein is low and comparable to that of healthy individuals, the individual from whose biological sample said first particular physiological protein was isolated is considered not to be affected by the chronic or acute pathology associated with nitrating stress in question. The individual from whose biological sample said first particular physiological protein was isolated may also be affected by the chronic or acute pathology associated with nitrating stress in a phase with little progression or in remission.

If the degree of nitration of the first nitrated protein is greater than zero, the individual is affected by the chronic or acute pathology associated with nitrating stress in question, and said chronic or acute pathology may be in the progressive phase or in the acute phase.

In an advantageous embodiment of the invention, the degree of nitration of the first particular protein can be compared with the degree of nitration of a third particular nitrated physiological protein. “Third physiological protein” defines a protein having a sequence identity of at least 90%, and more particularly 100%, with said first particular protein, said third protein being obtained from a biological sample, of the same nature or of a different nature, from an individual other than the individual from whom the first particular protein was obtained. In particular, the individual from whom the third particular nitrated protein was obtained corresponds to an individual who is affected by said pathology associated with nitrating stress, and presents all the symptoms associated with this pathology.

Thus, from the comparison of the degree of nitration of the first physiological protein with the degree of nitration of the second and/or of the third particular physiological protein, it is possible to determine the degree of nitrating stress, which can be correlated with the state of severity and progressiveness of the pathology, chronic or acute, associated with nitrating stress in the patient from whom the first physiological protein was obtained.

The invention also relates to a method of in vitro diagnosis characterized by the following stages:
quantitative assay, in a biological sample from an individual, of the degree of nitration of a first particular nitrated protein or physiological peptide sequence,
quantification of said degree of nitration of said first particular protein or physiological peptide sequence by comparing the concentration of said first particular nitrated protein or physiological peptide sequence with the total concentration of said first particular protein or physiological peptide sequence, nitrated and non-nitrated, obtained from a biological sample from the same individual,
comparing said degree of nitration of tyrosine residues of the first particular nitrated protein or physiological peptide sequence with the degree of nitration of a set of n particular proteins or physiological peptide sequences, the value of the degree of nitration of each of the aforesaid n particular proteins or peptide sequences being known and associated respectively with a particular state of severity and progressiveness of said chronic or acute pathology,
   said n particular proteins or physiological peptide sequences being variants or isoforms of the aforesaid first particular protein or peptide sequence, or having an amino acid sequence identity of at least 90%, and in particular 100%, with the aforesaid first particular protein or physiological peptide sequence,
   said n particular proteins or physiological peptide sequences being nitrated on at least one tyrosine residue located in an equivalent position to the tyrosine residue in the aforesaid first particular protein or physiological peptide sequence,
deduction, from the comparison carried out in the preceding stage, of the degree of nitrating stress of the individual, which may correspond to a state of severity and progressiveness of said chronic or acute pathology.

The n particular physiological proteins of the invention are defined such that they have sequence homology of at least 90%, and more particularly 100% identity with the first particular protein or that they are derived from the same gene. In the invention, n varies from 2 to 10, and advantageously from 2 to 5.

Said n proteins are obtained from n different biological samples. The degree of nitration of each of the n particular proteins is of known value and is associated with a particular state of said pathology associated with nitrating stress.

The method also described in the invention therefore consists of an assay the degree of physiological nitration of a first particular physiological protein of the invention, from a biological sample from an individual.

The previously determined degree of nitration is compared with the degree of nitration of a set of n particular proteins, each of the n particular proteins being obtained from a different biological sample, i.e. n biological samples. The n samples can be obtained either from n different individuals, or from the same individual, but from whom samples were taken in the course of the n stages of the pathology associated with nitrating stress that he has developed.

The n biological samples correspond to a different stage of development of the pathology associated with nitrating stress that is well characterized, and quantified.

In one of the advantageous embodiments of the invention, the n particular proteins are defined as follows: a particular protein corresponds to a protein obtained from a biological sample from an individual not affected by the pathology associated with nitrating stress, and the n−1 other particular proteins correspond to n−1 biological samples defining n−1 particular different states of severity and progressiveness of said pathology associated with nitrating stress.

The degrees of nitration of the first and of the second particular physiological protein are then compared.

When the degree of nitration of the first nitrated protein corresponds to the degree of nitration of one of the n particular physiological proteins the individual is affected by a nitrating stress associated with the chronic or acute pathology in question, to the same degree as that of the individual from whom the n particular nitrated physiological protein was obtained.

More particularly, the invention relates to a method of in vitro diagnosis in which the in vitro quantitative assay is carried out by means of at least one antibody, each antibody specifically recognizing a nitrated tyrosine residue of a particular nitrated protein or physiological peptide sequence.

Advantageously, the invention describes a method of diagnosis mentioned previously, in which the particular nitrated physiological protein is preferably a circulating protein, in particular in the plasma, in particular selected from: albumin, prealbumin, vitamin D binding protein (VDBP), transferrin, ceruloplasmin, retinol binding protein (RBP), insulin, haemoglobin, β actin, band 3 protein of erythrocyte anion transporter, β chain of erythrocyte spectrin, fibronectin precursor, β chain of fibrinogen and erythrocyte membrane protein band 4.1.

Moreover, the invention describes a method of diagnosis, in which the chronic or acute pathology associated with nitrating stress belongs to the following group: inflammatory diseases, infectious diseases, neurodegenerative diseases, hypoxic and ischaemic diseases, diabetes, metabolic disorders, cardiovascular and respiratory diseases, and cancer.

According to an advantageous embodiment, the invention describes a method of in vitro diagnosis of the state of severity and progressiveness of a chronic or acute pathology associated with nitrating stress, in a biological sample, comprising:
detection of an immune complex resulting from bringing at least one antibody specifically recognizing a nitrated tyrosine residue of physiological albumin into contact with nitrated physiological albumin obtained from a biological sample from an individual, said detection of the immune complex permitting determination of the degree of nitration of said nitrated physiological albumin,
comparing said degree of nitration of tyrosine residue of physiological albumin, with the degrees of nitration of tyrosine residues of a set of n physiological albumins the value of the degree of nitration of which is known and is associated respectively with a particular state of severity and progressiveness of said chronic or acute pathology,
deduction, from the comparison carried out in the preceding stage, of the degree of nitrating stress of the individual, which may correspond to a state of severity and progressiveness of said chronic or acute pathology.

The “immune complex” referred to above (or antigen-antibody complex) results from the combination of an epitope (antigen) with an antibody directed against this epitope and only against this antigen. The antigen involved in the invention corresponds to the particular nitrated proteins mentioned previously.

The immune complex is detected by means of monoclonal or polyclonal antibodies specifically recognizing the particular protein, directly or indirectly. In direct detection, said antibody permitting detection is generally coupled to labels. The labels can be selected from radiolabels, biotin, enzymes, fluorescing agents, magnetic particles, etc.

In indirect detection, the antibody recognizing the particular protein is itself recognized by a detecting antibody coupled to one of the labels.

The antibodies used can be used in soluble form, or immobilized on supports, and more particularly beads, plates, columns, etc.

The method described in the invention consists of an assay of the degree of physiological nitration of a first particular physiological protein, from a biological sample from an individual.

The previously determined degree of nitration is compared with the degree of nitration of a set of n particular proteins, each of the n particular proteins being obtained from a different biological sample, i.e. n biological samples. The n samples can be obtained either from n different individuals, or from the same individual, but from whom samples were taken in the course of the n stages of the pathology associated with nitrating stress that he has developed.

The n biological samples correspond to a different stage of development of the pathology associated with nitrating stress that is well characterized, and quantified.

In one of the advantageous embodiments of the invention, the n particular proteins are defined as follows: a particular protein corresponds to a protein obtained from a biological sample from an individual not affected by the pathology associated with nitrating stress, and the n−1 other particular proteins correspond to n−1 biological samples defining n−1 different particular states of severity and progressiveness of said pathology associated with nitrating stress.

When the degree of nitration of the first nitrated protein corresponds to the degree of nitration of one of the n particular physiological proteins, the individual is affected by the chronic or acute pathology associated with nitrating stress in question, at a stage identical to that of the individual from whom the n particular nitrated physiological protein was obtained.

More particularly, the invention relates to a method of in vitro diagnosis, in which said antibody recognizes the nitration of the albumin on the Y¹³⁸ tyrosine residue.

Similarly, the invention relates to a method of in vitro diagnosis, in which said antibody recognizes the nitration of the albumin on the Y⁴¹¹ tyrosine residue.

One aspect of the invention relates more particularly to a method of in vitro diagnosis of the state of severity and progressiveness of a chronic or acute pathology associated with nitrating stress, in a biological sample, comprising:
detection, by means of an antibody directed against the albumin, of an immune complex resulting from bringing at least one specific antibody into contact with nitrated physiological albumin obtained from a biological sample from an individual, said detection of the immune complex permitting the determination of the degree of nitration of the nitrated physiological albumin,
comparing said degree of nitration of tyrosine residue of nitrated physiological albumin, with the degrees of nitration of tyrosine residues of a set of n nitrated physiological albumins of which the value of the degree of nitration of tyrosine residues of nitrated physiological albumin is known and is linked respectively to a state of severity and progressiveness of said chronic or acute pathology,
deduction, from the comparison carried out in the preceding stage, of the degree of nitrating stress of the individual, which may correspond to a state of severity and progressiveness of said chronic or acute pathology.

Thus, the method described in the invention consists of an assay of the degree of physiological nitration of physiological albumin of the invention, from a biological sample from an individual, using the specific antibodies recognizing nitrated albumin of the invention.

The antibodies of the invention make possible the specific immobilization of nitrated albumin among a population of proteins, nitrated and non-nitrated. The immune complex formed is then detected and the quantity of complex is indicative of the degree of nitration of the albumin in the biological sample.

The previously determined degree of nitration is compared with the degree of nitration of a set of n albumins, each of the n albumins being obtained from a different biological sample. The n samples can be obtained either from n different individuals, or from the same individual, but from whom samples were taken in the course of the n stages of the pathology associated with nitrating stress that the patient has developed.

The n biological samples correspond to different stages of development of the pathology associated with nitrating stress that is well characterized, according to the clinical criteria of the pathology, and the degree of nitration of the albumin obtained from these n samples is quantified.

In one of the advantageous embodiments of the invention, the n particular albumins are defined as follows: a particular albumin corresponds to a protein obtained from a biological sample from an individual not affected by the pathology associated with nitrating stress, and the n−1 other particular albumins correspond to n−1 biological samples defining n−1 different particular states of severity and progressiveness of said pathology associated with nitrating stress.

The invention also relates to a method of in vitro diagnosis in which the degree of nitrating stress of the individual can correspond to a state of severity and progressiveness of the pathologies selected from: inflammatory diseases, infectious diseases, neurodegenerative diseases, ischaemic diseases, diabetes, metabolic disorders, cardiovascular and respiratory diseases, and cancer.

An advantageous method of in vitro diagnosis of the invention uses in particular the detection of the immune complex by means of conventional techniques such as ELISA (direct or competitive), immunohistochemistry and immunocytochemistry, immunoprecipitation, nephelometry, turbidimetry, Western blot or any other immunochemical or radio-immunological assay (RIA).

ELISA, RIA, immunohistochemistry and immunocytochemistry, immunoprecipitation, nephelometry, turbidimetry, Western blot or any other immunochemical method are standard techniques known by a person skilled in the art.

More particularly, the invention describes a method of diagnosis, preferably in vitro, of the state of severity and progressiveness of a chronic or acute pathology associated with nitrating stress in a biological sample from an individual, as defined previously, in which the quantitative assay, in a biological sample from an individual, of the degree of nitration of a first protein, in particular albumin, or particular nitrated physiological peptide sequence, is carried out by means of at least one antibody as defined previously, said antibody either being fixed on a support as a capture antibody, or serving as a specific detecting antibody.

In other words the aforementioned method of diagnosis is preferably carried out by ELISA, in which
either a specific antibody of nitrated or non-nitrated albumin is immobilized and serves as a capture antibody for nitrated or non-nitrated albumin, and the immobilized nitrated proteins are revealed by means of the monoclonal antibody as defined above; optionally, the two monoclonal antibodies of the invention can be used simultaneously,
or a monoclonal antibody, as defined above, specific to nitrated albumin is immobilized and serves as a capture antibody for nitrated albumin, and the immobilized nitrated proteins are revealed by means of an antibody recognizing nitrated or non-nitrated albumin, optionally, the two monoclonal antibodies of the invention can be used simultaneously,
or a monoclonal antibody as defined, specific to nitrated albumin, is immobilized and serves as a capture antibody for nitrated albumin, and the immobilized nitrated proteins are revealed by means of a monoclonal antibody as defined, specific to nitrated albumin.

In the last category, the possible pairs are
capture antibody: 2F3, detecting antibody: 13H10, or
capture antibody: 13H10, detecting antibody: 2F3.

Furthermore, in another preferred embodiment, the invention describes a method of diagnosis mentioned previously which corresponds to a radioimmunoassay (RIA) in which:
the sample obtained from the patient, which may contain nitrated albumin, is incubated with a known, defined quantity of nitrated albumin labelled with a tracer, said tracer being radioactive iodine (¹²⁵I), to obtain a mixture,
the aforementioned mixture is contacted with at least one of the specific antibodies of nitrated albumin defined above, to permit the formation of an immune complex,
the immune complex formed between said labelled nitrated albumin and at least one of the specific antibodies of nitrated albumin obtained in the next stage is detected and quantified by means of a specific system for detecting the labelling of said nitrated albumin, said detection preferably being carried out by means of a gamma counter, or any counter for detecting the disintegration of radioactive isotopes,
deduction, from the preceding quantification, of the quantity of nitrated albumin initially present in the sample.

In RIA, the antigen to be assayed (nitrated albumin) competes with the labelled antigen (labelled nitrated albumin) for binding to the antibody; all the available antibody sites are bound. The bound fraction, which decreases exponentially with the concentration of antigen to be assayed, is measured. The procedure can be carried out in a homogeneous liquid phase or in a heterogeneous solid phase; in the latter case, it is easier to separate the free and bound fractions.

The principle of assay by RIA is based on competition between nitrated albumin labelled with iodine 125 and nitrated albumin contained in standards or the samples to be measured, for a given, limited number of specific anti-nitrated albumin antibody sites (13h10 and/or 2F3 Antibody) optionally fixed on a solid phase (coated tubes, microtitre plate etc.). At the end of the incubation time, the excess of tracer is easily removed in a washing stage. The quantity of labelled nitrated albumin bound to the antibody is inversely proportional to the quantity of unlabelled nitrated albumin present in the test.

The present invention also relates to the use of a particular nitrated protein or peptide sequence, nitrated on at least one tyrosine residue, for the implementation of a method of quantitative assay, in particular in vitro, of the degree of physiological nitration of a protein or of a nitrated physiological peptide sequence, said particular nitrated protein or peptide sequence having an amino acid sequence identity of at least 90%, and in particular 100%, with the aforesaid particular nitrated protein or physiological peptide sequence, said particular nitrated protein or peptide sequence having nitration on at least one tyrosine residue in an equivalent position to that of the tyrosine residue of the aforesaid particular nitrated protein or physiological peptide sequence, the nitration of said particular protein or physiological peptide sequence being correlated with the nitrating stress associated with chronic or acute pathologies.

The invention relates more particularly to the use of a particular nitrated protein or peptide sequence, in which said particular nitrated protein or peptide sequence makes it possible to generate at least one monoclonal or polyclonal antibody capable of specifically recognizing a nitrated tyrosine residue of said particular protein or peptide sequence, said antibody also being capable of specifically recognizing said nitrated tyrosine residue in an equivalent position in the particular nitrated protein or physiological peptide sequence.

The invention also relates to a method of in vitro preparation of a particular nitrated protein or peptide sequence on tyrosine residues in which the nitrated tyrosine residues correspond to the physiologically nitrated tyrosine residues of a particular nitrated protein or physiological peptide sequence in chronic or acute pathologies associated with nitrating stress, said particular nitrated protein or peptide sequence having an amino acid sequence identity of at least 90%, and in particular 100%, with the aforesaid particular nitrated physiological protein, comprising:
in vitro nitration of a particular protein or peptide sequence by tetranitromethane in a molar ratio with the nitratable tyrosines of the particular protein or peptide sequence not exceeding 20:1, in an aqueous buffer with pH between 7.5 and 8.5,
in vitro identification of the nitrated tyrosines of said particular nitrated protein, in particular by mass spectrometry, and
optionally, comparison of the nitrated tyrosines identified on said particular protein or peptide sequence with the tyrosine residues of the particular nitrated protein or physiological peptide sequence.

There are numerous agents for nitrating proteins, in particular peroxynitrite, tetranitromethane, 3-morpholino-sydonimine, sodium α-oxyhyponitrite, spermine-NONOate and other NONOates, nitryl chloride (NO₂Cl), nitroprusside and nitroglycerin. These nitrating agents are not the only ones. The above list of nitrating agents is not exhaustive.

The invention has the advantage of providing a method for specific in vitro nitration of the proteins on tyrosine residues, said residues being nitratable physiologically.

In contrast to what is described in the prior art, the method of the invention uses tetranitromethane under conditions of low concentrations which can be regarded as simulating conditions close to the physiological conditions of nitration. The advantage of in vitro nitration by tetranitromethane in an aqueous medium at weakly alkaline pH relative to peroxynitrite is that, when used under these conditions, tetranitromethane is far less oxidizing than peroxynitrite, thus avoiding the formation of oxidized derivatives and amino acid oxidation products, in particular cysteines, methionines, tryptophans and tyrosines, of the proteins treated. This method also minimizes the formation of disulphide bridges as well as dityrosines.

The method of the invention therefore describes a stage of nitration of proteins, in which the quantities of tetranitromethane depend on the quantity of nitratable tyrosines contained in the protein to be nitrated. In order to adjust the concentrations of tetranitromethane to be used in the method of the invention, it is therefore necessary to estimate or determine the number of nitratable or nitrated tyrosines of the proteins in question.

One means of determining the nitratable or nitrated tyrosines of the proteins is to perform an in vitro nitration of the proteins with tetranitromethane, at increasing molar ratios relative to the number of tyrosine residues present in the protein. At the end of nitration, the protein is purified on agarose-conjugated anti-nitrotyrosine. The nitrated residues are then determined by mass spectrometry. This method provides information relating to the number of nitratable tyrosines.

The following diagrams illustrate the invention:

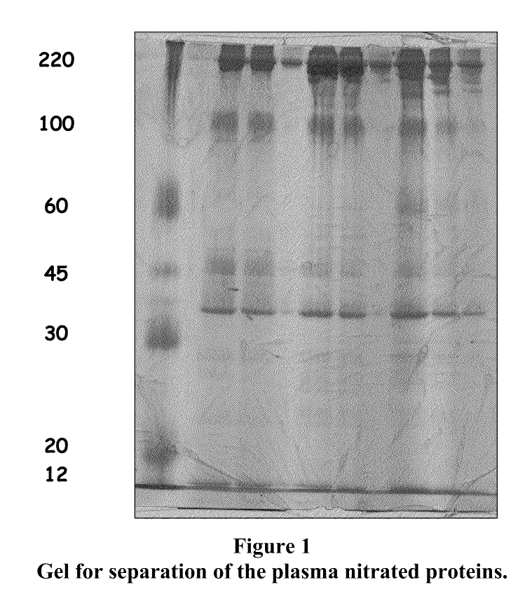

FIG. 1 shows a gel for separating nitrated proteins obtained from plasma samples. The plasma samples are subjected to immunoprecipitation with an agarose-conjugated anti-nitrotyrosine antibody. The immunoprecipitated nitrated proteins are separated by electrophoresis (SDS-PAGE) and revealed by staining with colloidal Coomassie Blue. The lanes correspond to immunoprecipitates of nitrated proteins from plasma from different patients.

Molecular weight markers are indicated on the left-hand part of the gel.

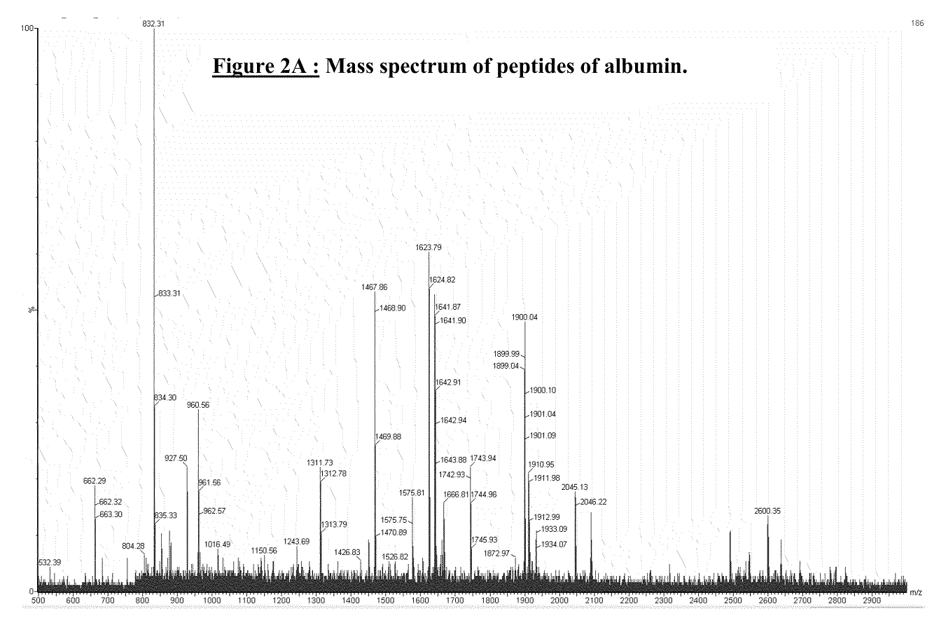

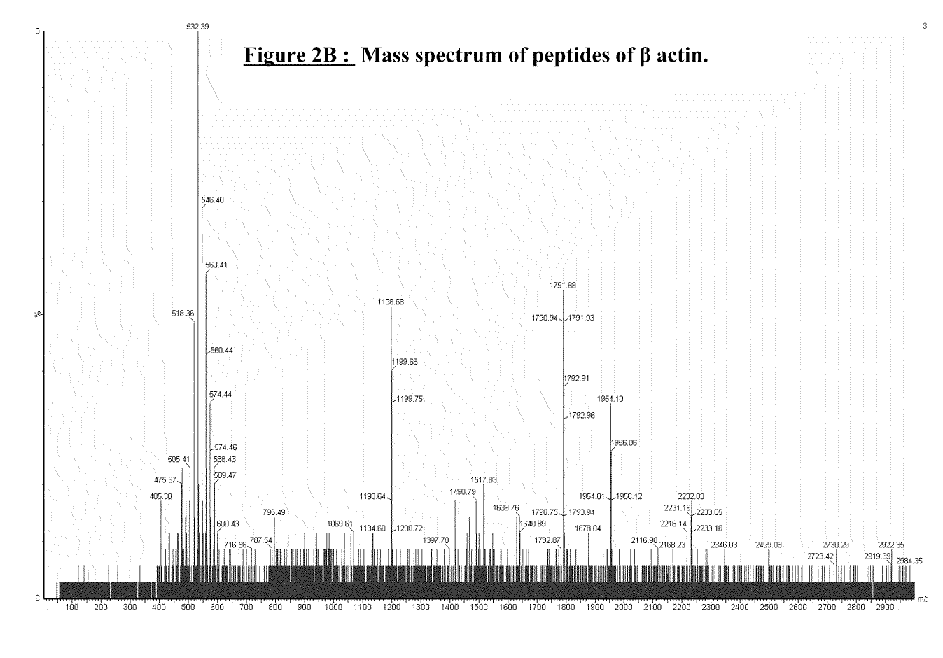

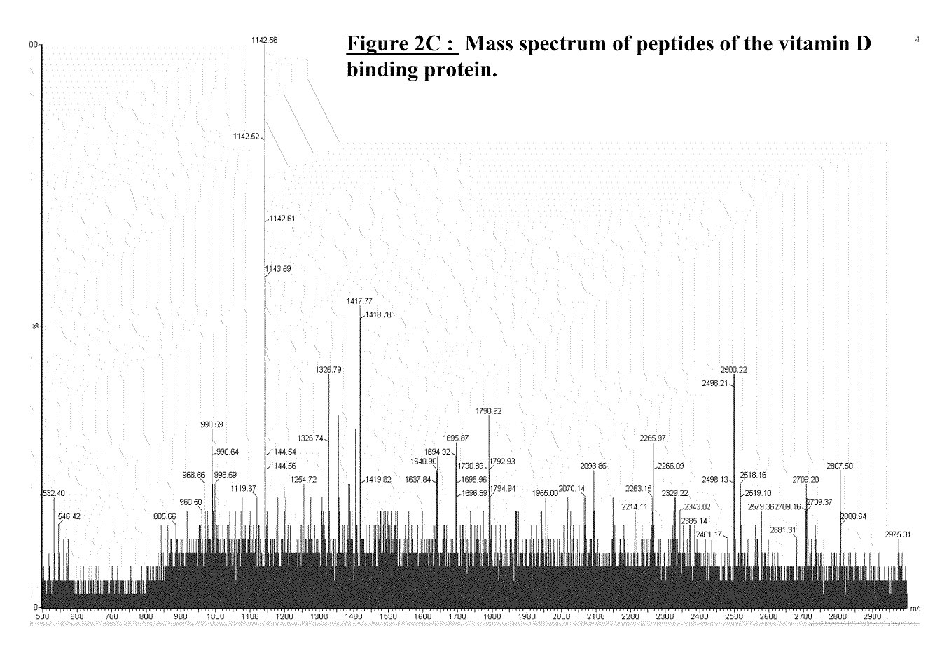

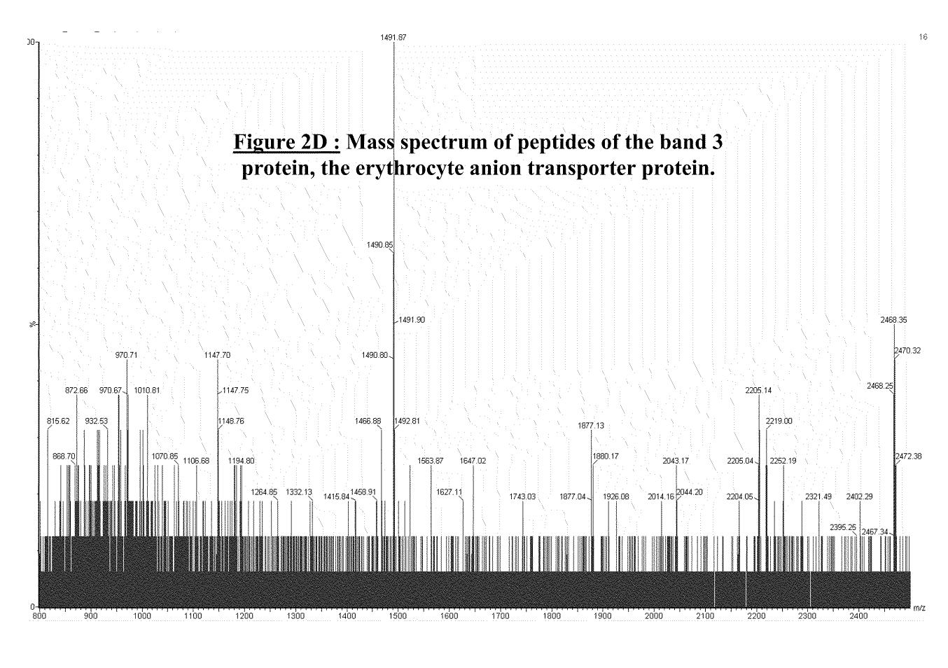

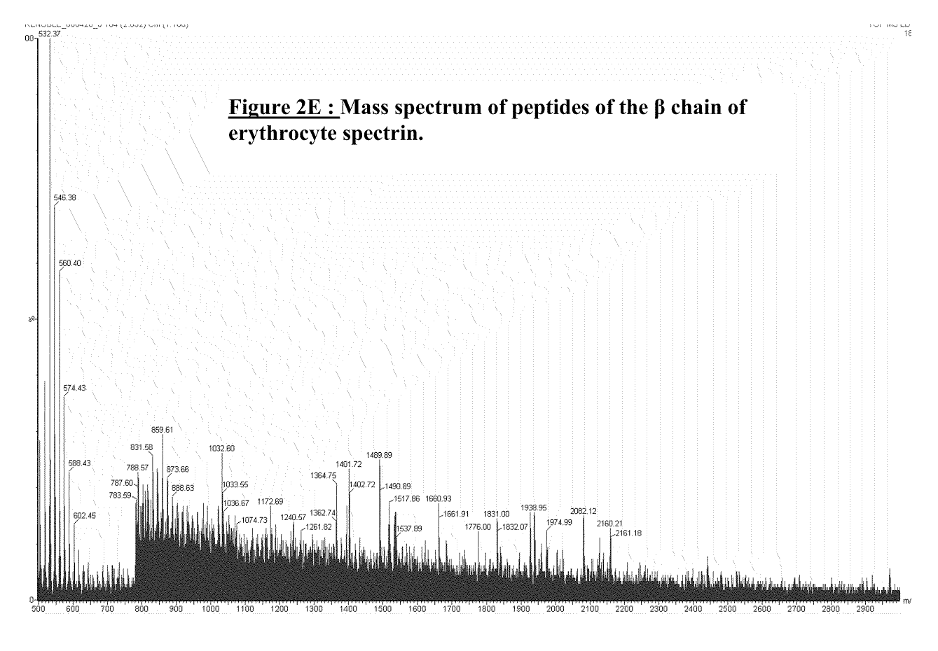

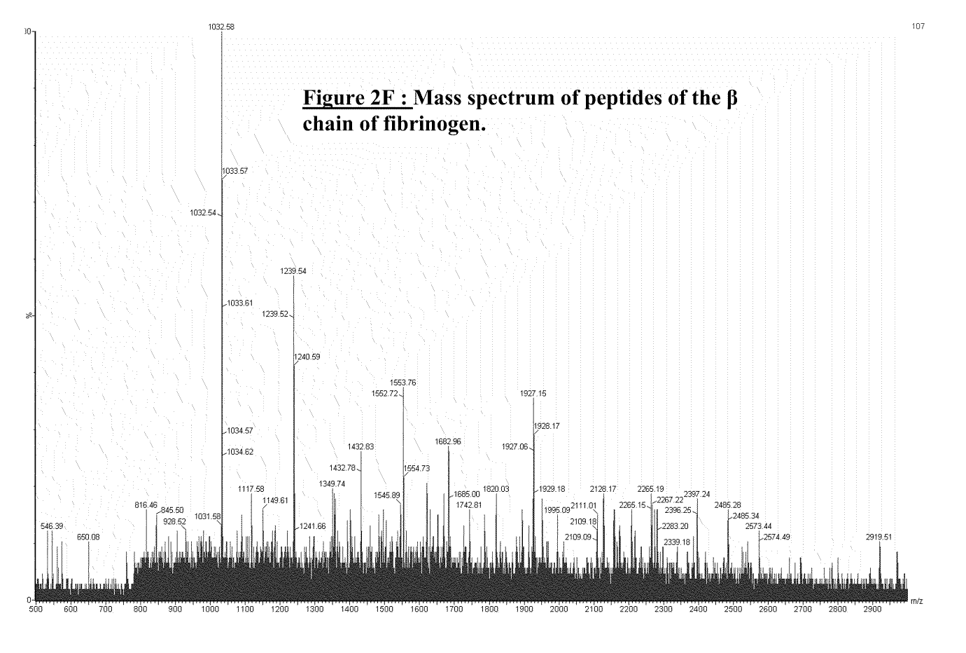

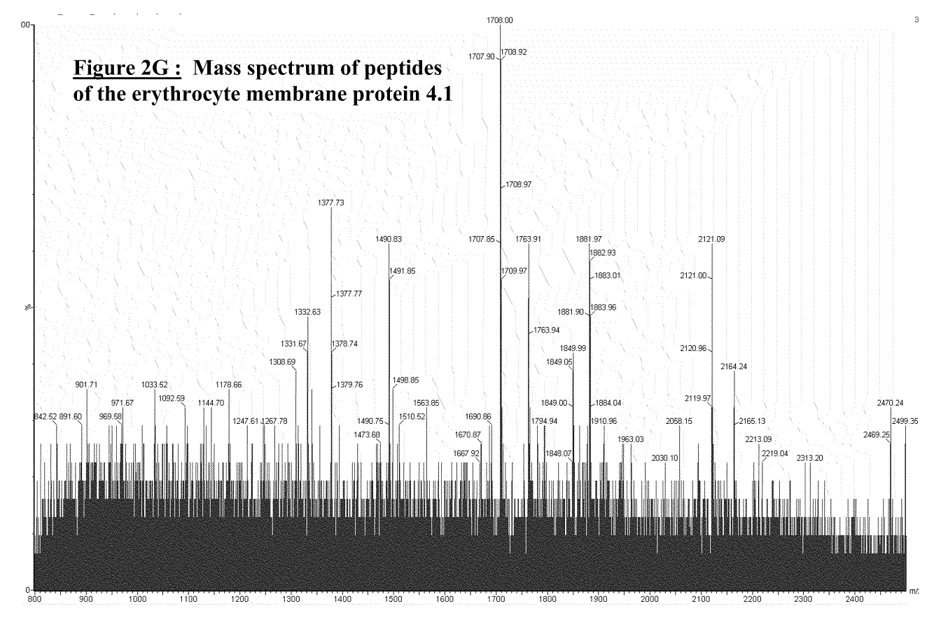

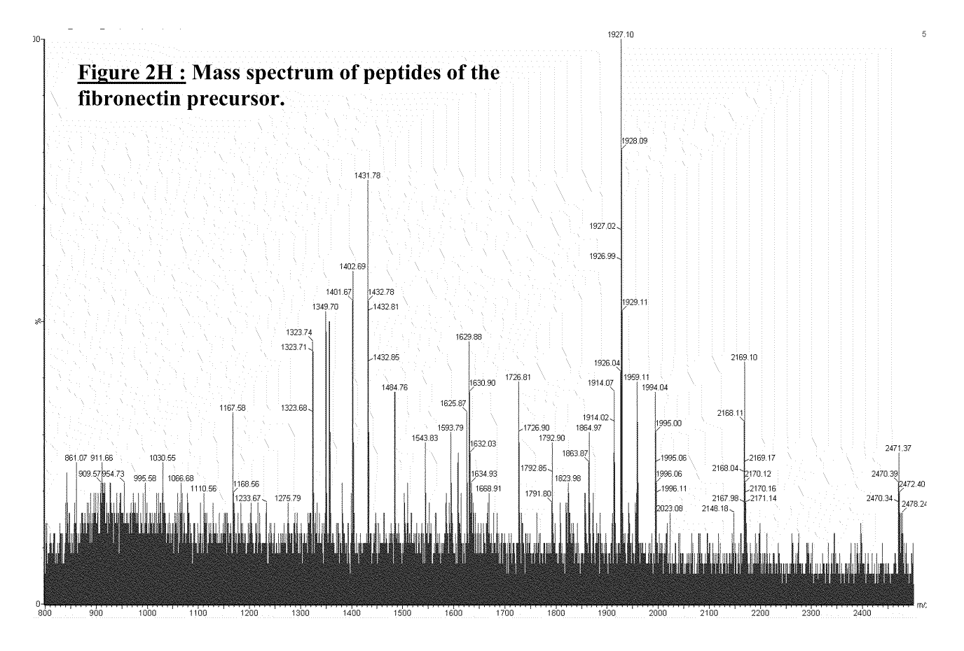

FIGS. 2A, 2B, 2C, 2D, 2E, 2F, 2G and 2H show the mass spectra of the nitrated proteins taken from the separating gel. The mass spectra of the peptides are obtained by the MALDI-TOF method.

FIG. 2A corresponds to the mass spectrum corresponding to the peptides obtained from albumin.

FIG. 2B corresponds to the mass spectrum corresponding to the peptides obtained from β actin.

FIG. 2C corresponds to the mass spectrum corresponding to the peptides obtained from vitamin D binding protein.

FIG. 2D corresponds to the mass spectrum corresponding to the peptides for identifying band 3 of the erythrocyte anion transporter protein.

FIG. 2E corresponds to the mass spectrum corresponding to the peptides obtained from the β chain of erythrocyte spectrin.

FIG. 2F corresponds to the mass spectrum corresponding to the peptides obtained from the β chain of fibrinogen.

FIG. 2G corresponds to the mass spectrum corresponding to the peptides obtained from erythrocyte membrane protein band 4.1.

FIG. 2H corresponds to the mass spectrum corresponding to the peptides obtained from fibronectin precursor.

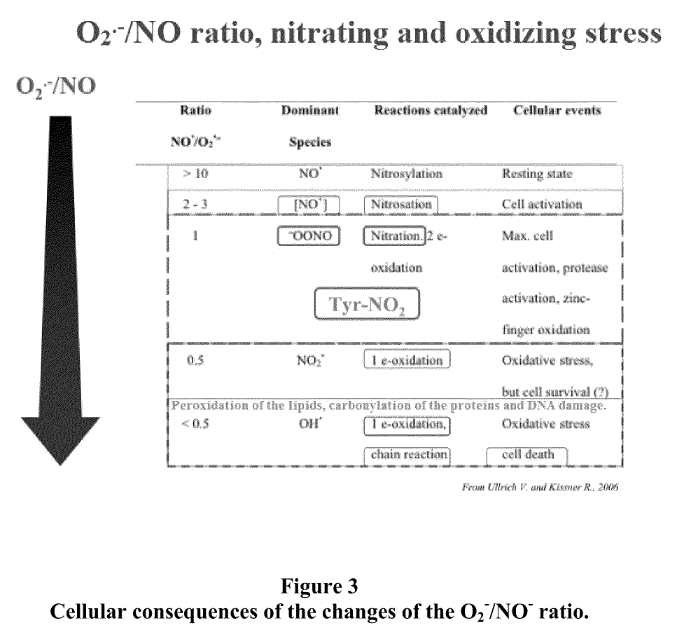

FIG. 3 is a schematic representation of the cellular consequences of changes in the O₂.⁻/NO. molar ratio.

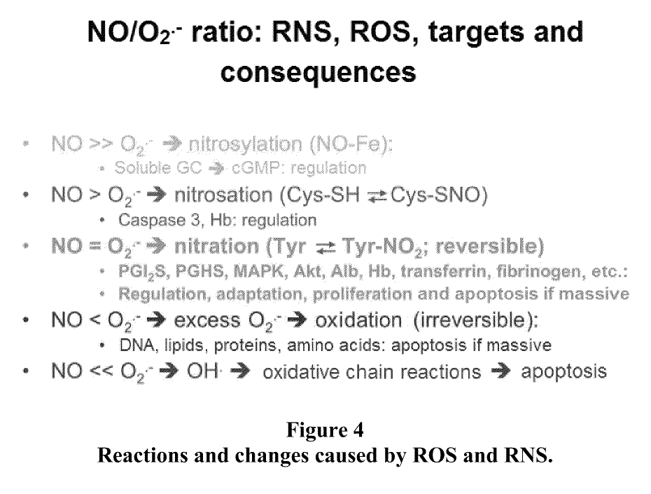

FIG. 4 shows the post-translational reactions and modifications caused by the ROS and RNS formed as a function of the NO.^{/}O₂.⁻ molar ratio.

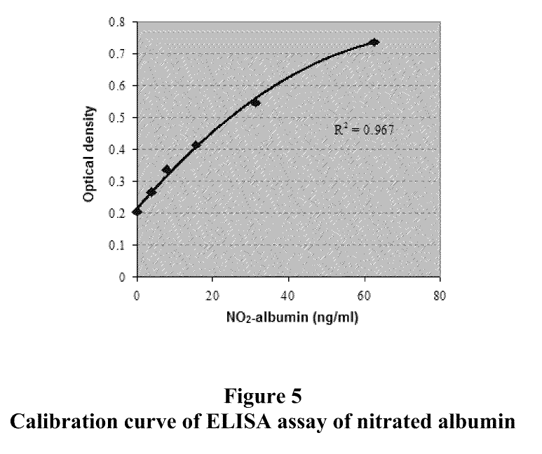

FIG. 5 shows the calibration curve of ELISA assay of nitrated albumin. This assay uses an anti-nitrotyrosine polyclonal capture antibody and an anti-human albumin detecting antibody.

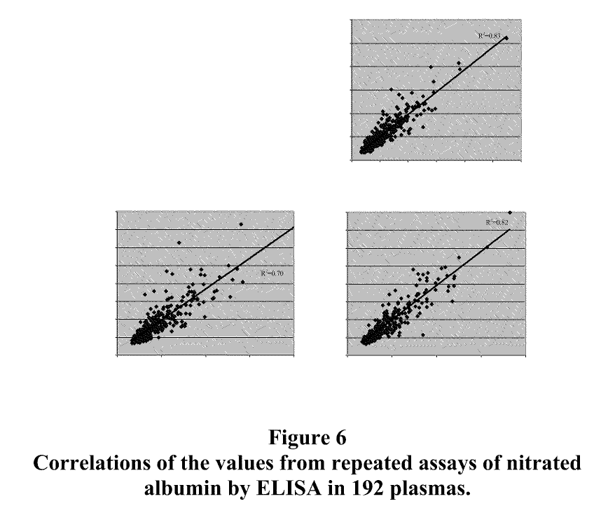

FIG. 6 shows the correlations between the values obtained for the assays of nitrated albumin, carried out in triplicate, in 192 plasmas from neonates aged from 0 to 5 days.

FIG. 7 shows the dose-response curve for detection of nitrated albumin in ELISA in which the 13H10 antibody is used as capture antibody and an anti-human albumin polyclonal antibody conjugated with peroxidase (HRP) is used as detecting antibody. The abscissa (log(x)) shows the concentration of albumin and the ordinate shows the optical density (OD) at 450 nm.

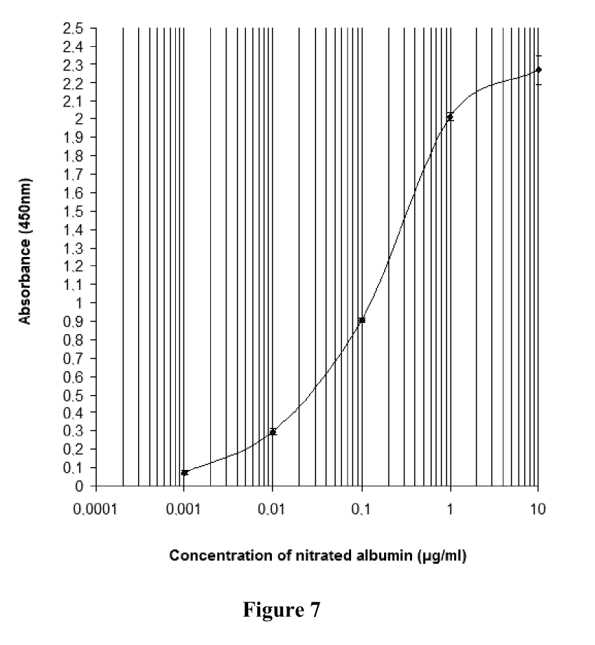

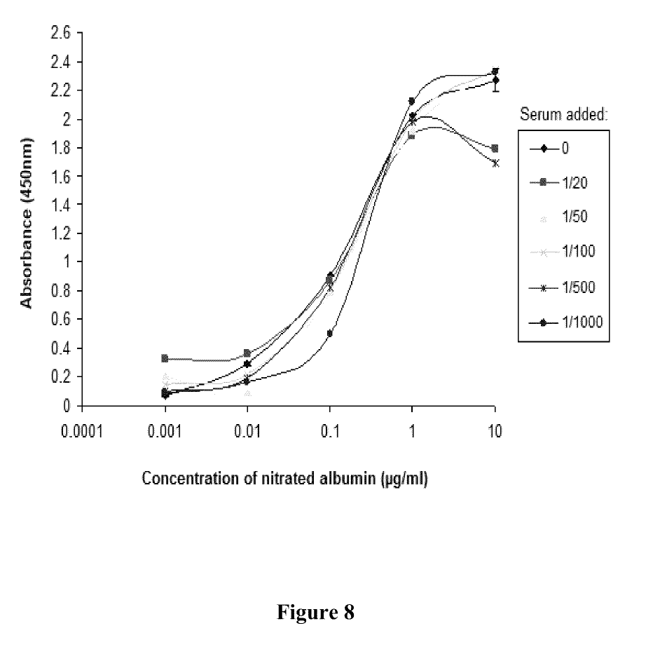

FIG. 8 shows the optical density (OD) measured at 450 nm as a function of the nitrated albumin concentration in ELISA in which the 13H10 antibody is used as capture antibody and an anti-human albumin polyclonal antibody conjugated with peroxidase (HRP) is used as detecting antibody and human serum. The abscissa (log(x)) shows the nitrated albumin concentration and the ordinate shows the optical density (OD) at 450 nm.

The quantities of serum added are also shown in the figure.

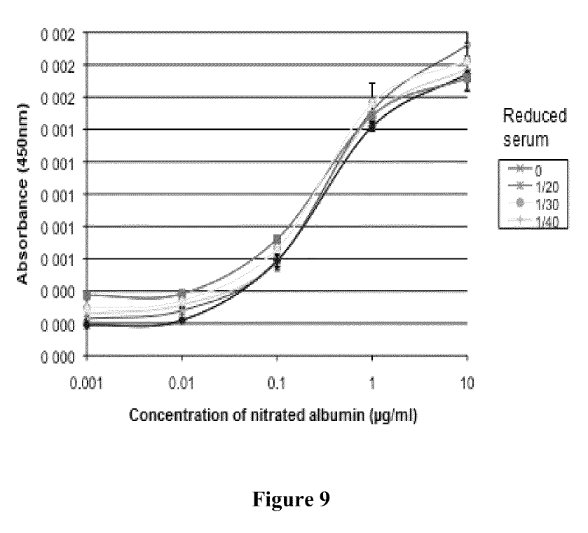

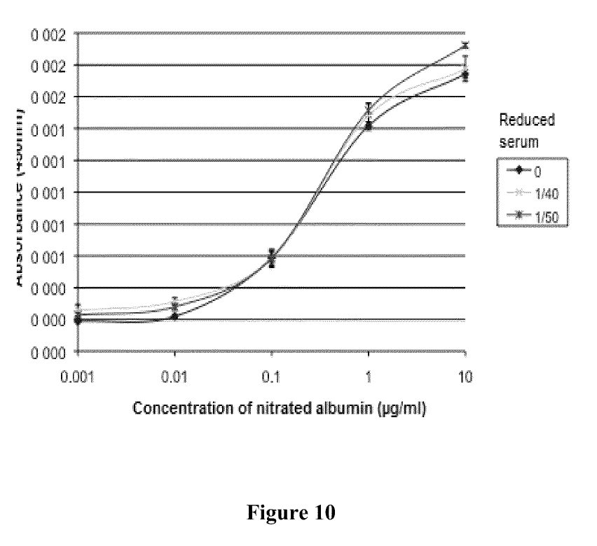

FIGS. 9 and 10 show the optical density (OD) measured at 450 nm as a function of the nitrated albumin concentration in ELISA in which the 13H10 antibody is used as capture antibody and an anti-human albumin polyclonal antibody conjugated with peroxidase (HRP) is used as detecting antibody and in the presence of reduced human serum. The abscissa (log(x)) shows the nitrated albumin concentration and the ordinate shows the optical density (OD) at 450 nm.

The quantities of serum added are also shown in the figures.

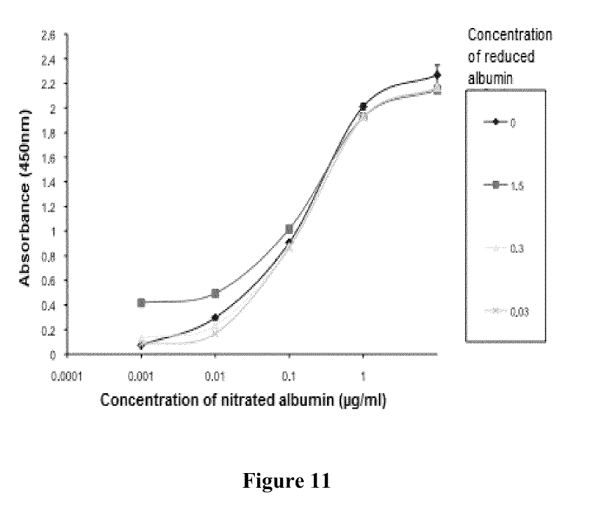

FIG. 11 shows the optical density (OD) measured at 450 nm as a function of the nitrated albumin concentration in ELISA in which the 13H10 antibody is used as capture antibody and an anti-human albumin polyclonal antibody conjugated with peroxidase (HRP) is used as detecting antibody and in the presence of reduced human albumin. The abscissa (log(x)) shows the nitrated albumin concentration and the ordinate shows the optical density (OD) at 450 nm.

The concentrations of reduced human albumin added are also shown in the figure.

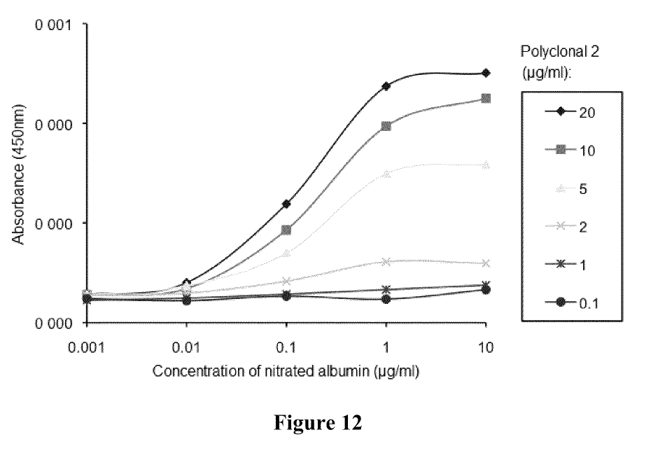

FIG. 12 shows the absorbance as a function of the nitrated albumin concentration in ELISA in which an anti-human albumin polyclonal antibody is used as capture antibody and the 13H10 antibody conjugated with peroxidase (HRP) is used as detecting antibody. The abscissa (x) shows the nitrated albumin concentration and the ordinate shows the optical density (OD) at 450 nm.

The quantities of polyclonal antibody added for detection are also shown in the figure.

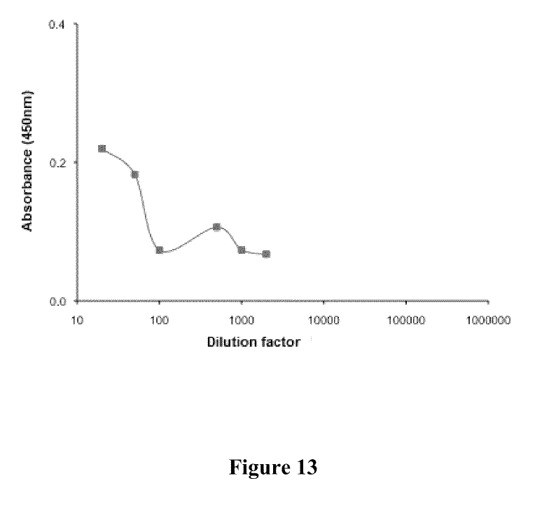

FIG. 13 shows the absorbance as a function of the dilution factor of the 13H10 antibody for the detection of nitrated albumin in samples of human serum. The abscissa (x) shows the dilution factor of the antibody and the ordinate shows the optical density (OD) at 450 nm.

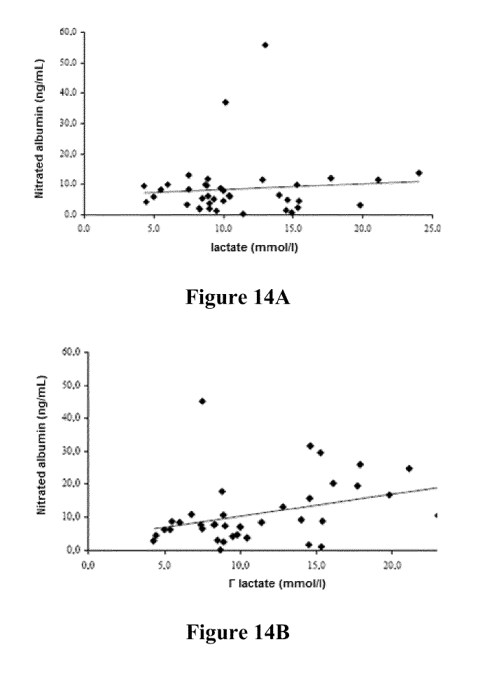

FIGS. 14A and B show the correlation between arterial lactacidaemia and the plasma nitrated albumin concentration during the first hours of life (FIG. 14A) and at D1 (FIG. 14B).

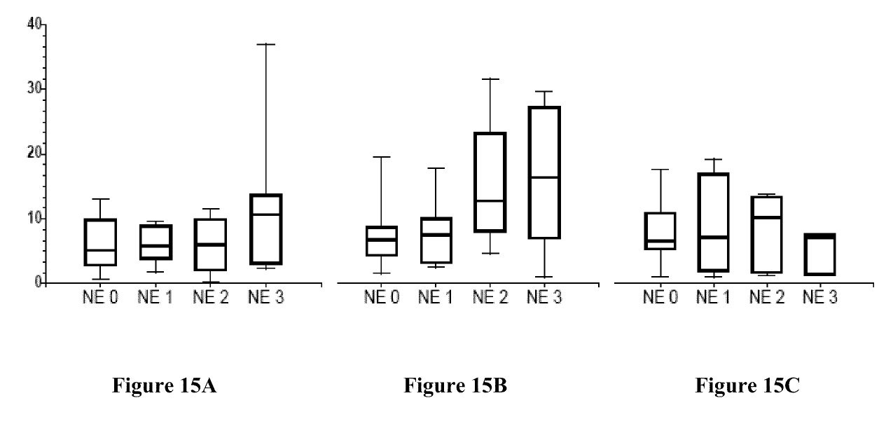

FIGS. 15 A-C show the nitrated albumin concentrations (expressed in medians, 25th-75th percentiles and 10th-90th percentiles) during the first hours of life (FIG. 15A), at D1 (FIG. 15 B) and at D4 (FIG. 15 C), corresponding to the neurological status of neonates (normal (NE 0) or mild NE (NE 1) versus moderate (NE 2) to severe NE (NE 3)).

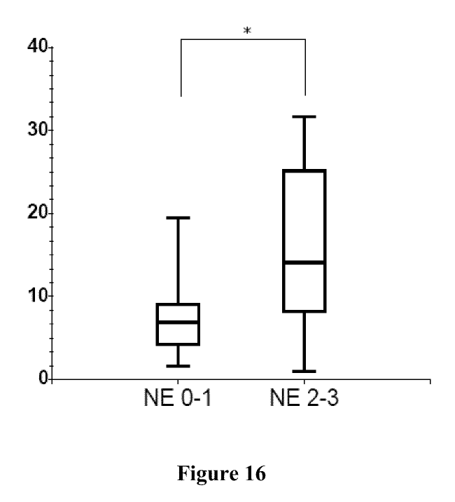

FIG. 16 shows the nitrated albumin concentration at day D1 (expressed in median, 25th-75th percentiles and 10th-90th percentiles) corresponding to the neurological status of neonates (normal (NE 0) or mild NE (NE 1) versus moderate (NE 2) to severe NE (NE 3)). The asterisk represents a significance value p=0.01

### EXAMPLES

### Experimental Part

### Example 1

### Nitration of Albumin In Vitro

### Materials:

Human albumin (>98%, Fluka)

### Tetranitromethane (Aldrich)

### Tris (Sigma)

### Dimethylsulphoxide (>99.5%, Fluka)

### Method:

The albumin is dissolved in a solution of Tris (50 mM, pH 8.0) at a concentration of 0.1 mM.

The tetranitromethane is dissolved in dimethylsulphoxide at a concentration of 800 mM and stored at −20° C.

Tetranitromethane dissolved in dimethylsulphoxide is added to the solution of albumin while stirring, in order to obtain a final concentration of 2 mM.

This solution is incubated at 20° C. for 12 h while stirring gently, in the dark.

After nitration, the solution is frozen at −80° C. and lyophilized in order to remove the residual tetranitromethane and ensure optimum preservation of the nitrated albumin.

The method of nitration was modified from Malan, P. G., et al. (1970) Biochemistry 9(16), 3205-3214 and Sokolovsky, et al. (1966) Biochemistry 5(11), 3582-3589.

### Example 2

### Nitration of Insulin In Vitro

### Materials:

Bovine insulin (I 1882, Sigma, ≧25 USP units/mg)

### Tetranitromethane (Aldrich)

### Tris (Sigma)

### Dimethylsulphoxide (>99.5%, Fluka)

### Method:

The insulin is dissolved in a solution of Tris (50 mM, pH 8.0) to a concentration of 1 mM. The tetranitromethane is dissolved in absolute ethanol to a concentration of 800 mM and stored at −20° C.

Tetranitromethane dissolved in dimethylsulphoxide is added to the solution of insulin, while stirring, in order to obtain a final concentration of 20 mM.

This solution is incubated at 20° C. for 12 h, stirring gently in the dark (adapted from Morris et al. Biochemistry, 1970, 9(20) 3930-3937 and Carpenter et al. Biochemistry, 1980, 19(25) 5926-5931.

After nitration, the solution is frozen at −80° C. and lyophilized in order to remove the residual tetranitromethane and ensure optimum preservation of the nitrated insulin.

### Example 3

### Nitration of Haemoglobin In Vitro

### Materials:

Bovine haemoglobin (H2500 Sigma)

### Tetranitromethane (Aldrich)

### Tris (Sigma)

### Dimethylsulphoxide (>99.5%, Fluka)

### Method:

The haemoglobin is dissolved in phosphate buffer 75 mM/carbonate 25 mM, pH 7.5 saturated with CO₂, to a concentration of 0.1 mM.

The tetranitromethane is dissolved in absolute ethanol to a concentration of 800 mM and stored at −20° C.

Tetranitromethane dissolved in dimethylsulphoxide is added to the solution of haemoglobin while stirring, in order to obtain a final concentration of 4 mM.

This solution is incubated at 20° C. for 12 h under gentle stirring in the dark.

After nitration, the solution is frozen at −80° C. and lyophilized in order to remove the residual tetranitromethane and ensure optimum preservation of the nitrated haemoglobin.

The method of nitration was modified from Pietraforte, D., et al. (2003) Amino Acids 25(3-4), 341-350, Pietraforte, D., et al. (2001) Biochemistry 40(50), 15300-15309 and Minetti, M., et al. (2000) Biochemistry 39(22), 6689-6697.

### Example 4

### Detection of Nitrated Plasma Proteins

FIGS. 1 and 2 illustrate this example

### Materials:

Human plasma
Polyclonal anti-nitrotyrosine affinity-purified on 3-nitrotyrosine column

### CarboLink Gel and AminoLink Gel (Pierce)

### Plasma Samples

The samples of blood were collected in an EDTA tube within the scope of a study approved by the relevant Ethics Committee, either for assessment of healthy individuals or for diagnostic assessment of individuals who have suffered from asphyxia.

The samples were preserved in ice and centrifuged at 4° C. within an hour. The plasmas obtained were stored at −20° C. before being analysed. All the samples were analysed less than 6 months after they were taken.

### Immunoprecipitation of Nitrated Proteins

The rabbit anti-nitrotyrosine polyclonal antibody obtained by immunization with nitrated KLH is purified by affinity on a column of 3-nitrotyrosine coupled to agarose (AminoLink) by the amine group.

The purified antibody is then coupled covalently to agarose via its oxidized carbohydrate residues (carbonyls) with hydrazide groups grafted on agarose (CarboLink). The hydrazone bonds thus formed are stabilized by reduction with sodium cyanoborohydride (NaCNBH₃).

The plasma samples are incubated for 14 h at 4° C. stirring gently with anti-nitrotyrosine-agarose beads and then washed 6 times with PBS/Triton X-100 0.1%.

The beads are taken up in an equivalent volume of Laemmli buffer 2×, heated for 5 min at 60° C. and loaded on a 10% SDS polyacrylamide gel with a thickness of 1.0 mm.

Molecular weight markers are loaded so as to be able to estimate the mass of the proteins detected.

At the end of migration, the gel is fixed and stained with colloidal Coomassie Blue (cf. FIG. 1).

### Identification of the Proteins Present on the Gel

The bands visualized by staining with colloidal Coomassie Blue are cut out and frozen at −80° C.

They are then digested with trypsin and analysed by mass spectrometry (MALDI-QTOF). The mass spectra, permitting identification of the peptides of the proteins identified, are shown in FIGS. 2A, 2B, 2C, 2D, 2E, 2F, 2G and 2H.

In the plasma samples, the inventors found albumin, vitamin D binding protein (VDBP), actin, the band 3 protein of the erythrocyte anion transporter, the β chain of erythrocyte spectrin, fibronectin precursor+the β chain of fibrinogen and band 4.1 of erythrocyte membrane protein.

### Interpretation

The results provide visualization and identification of these nitrated proteins, in the form of coloured bands, in the plasma of these subjects. It can be seen that the intensity of the bands varies depending on the subjects, indicating a variable degree of nitration of the proteins from one subject to another.

Among the proteins that can be seen on the gel, the following could be identified by mass spectrometry: albumin, vitamin D binding protein (VDBP), β actin, band 3 protein of erythrocyte anion transporter, β chain of erythrocyte spectrin, fibronectin precursor+the β chain of fibrinogen and band 4.1 of erythrocyte membrane protein.

### Example 5

### Evaluation of the Nitration of Albumin in Patients with Heart Failure

### Introduction:

It has been demonstrated that heart failure (HF) is accompanied by an increase in oxidative stress and that increase of this stress correlates with the stage of the disease [Sorescu, D. and K. K. Griendling, Reactive oxygen species, mitochondria, and NAD(P)H oxidases in the development and progression of heart failure. Congest Heart Fail, 2002. 8(3): p. 132-40; White, M., et al., Increased systemic inflammation and oxidative stress in patients with worsening congestive heart failure: improvement after short-term inotropic support. Clin Sci (Lond), 2006. 110(4): p. 483-9; Mallat, Z., et al., Elevated levels of 8-iso-prostaglandin F2alpha in pericardial fluid of patients with heart failure: a potential role for in vivo oxidant stress in ventricular dilatation and progression to heart failure. Circulation, 1998. 97(16): p. 1536-9; Dhalla, A. K., M. F. Hill, and P. K. Singal, Role of oxidative stress in transition of hypertrophy to heart failure. J Am Coll Cardiol, 1996. 28(2): p. 506-14; Ferrari, R., et al., Oxidative stress during myocardial ischaemia and heart failure. Eur Heart J, 1998. 19 Suppl B: p. B2-11]. It seems clear that hypoperfusion and relative hypoxia of the tissues, resulting from HF, induce oxidative stress. However, the notion that an increase in oxidative stress could be the cause of progression of HF, or at least contribute to it, is gaining ground. This oxidative stress could, as for other chronic diseases, be of inflammatory systemic origin [Cotter, G., et al., Acute heart failure: a novel approach to its pathogenesis and treatment. Eur Heart Fail, 2002. 4(3): p. 227-34; Mann, D. L., Inflammatory mediators and the failing heart: past, present, and the foreseeable future. Circ Res, 2002. 91(11): p. 988-98; Yndestad, A., et al., Systemic inflammation in heart failure—the whys and wherefores. Heart Fail Rev, 2006. 11(1): p. 83-92; Tousoulis, D., et al., Statins in heart failure. Beyond the lipid lowering effect. Int J Cardiol, 2007. 115(2): p. 144-50].

Nitrosation induced by NO⁺ and nitration caused by ONOO⁻ are therefore events that occur before oxidation. These changes lead, as we have shown for the MAP kinases and PKB/Akt, to functional changes at the level of enzymes and structural proteins and hence metabolic and trophic effects [Frein, D., et al., Redox regulation: a new challenge for pharmacology. Biochem Pharmacol, 2005. 70(6): p. 811-23; Ullrich, V. and R. Kissner, Redox signaling: bioinorganic chemistry at its best. J Inorg Biochem, 2006. 100(12): p. 2079-86; Pinzar, E., et al., Angiotensin II induces tyrosine nitration and activation of ERK1/2 in vascular smooth muscle cells. FEBS Lett, 2005. 579(22): p. 5100-4; Lokuta, A. J., et al., Increased nitration of sarcoplasmic reticulum Ca2+-ATPase in human heart failure. Circulation, 2005. 111(8): p. 988-95] (FIG. 4).

As the other two markers available for nitrating stress, plasma nitrotyrosine and urinary NHPA, are not reliable [Ryberg, H. and K. Caidahl, Chromatographic and mass spectrometric methods for quantitative determination of 3-nitrotyrosine in biological samples and their application to human samples. J Chromatogr B Analyt Technol Biomed Life Sci, 2007; Pannala, A. S., et al., pH-dependent nitration of para-hydroxyphenylacetic acid in the stomach. Free Radic Biol Med, 2006. 41(6): p. 896-901], nitrated albumin therefore appears to constitute a novel, specific and unique marker of this stress and might on this basis reflect the capacity of the organism to deal with the oxidative stress to which it is exposed. In order to verify this hypothesis, a new potential plasma marker of nitration is proposed, nitrated albumin, determination of which by ELISA has been developed (FIGS. 5 and 6).

The population of interest within the scope of this study is that of patients with heart failure representative of the general population of the two regions participating in the project: Auvergne and Rhône-Alpes.

For this, on the one hand we have access to the outpatients of the RESIC38 cohort (about 450 patients) and on the other hand to those of the day hospitals (Grenoble, Lyons, Clermont-Ferrand) to cover the different stages of heart failure as defined by the NYHA (New York Heart Association, accessible on http:/www.resic38.org/doc/Documents/Classification-nyha.pdf). Healthy controls will be recruited through the CIC of Grenoble. It seems to us to be essential to extend this study to a representative population which will accordingly include many elderly patients and patients with co-morbidities, but the results of which will therefore be applicable in current medical practice.

To date, only two prognostic biological markers have been validated in heart failure: BNP or its precursor proNT-BNP as well as troponin T. Other biological parameters, in particular markers of inflammation have been investigated recently and seem to indicate the existence of an inflammatory syndrome in heart failure [Yndestad, A., et al., Systemic inflammation in heart failure—the whys and wherefores. Heart Fail Rev, 2006. 11(1): p. 83-92]. The hypothesis of the existence of a chronic inflammatory syndrome associated with a certain number of risks factors and systemic chronic pathologies was the object of a commentary in The Lancet in 2007 [Fabbri, L. M. and K. F. Rabe, From COPD to chronic systemic inflammatory syndrome? Lancet, 2007. 370(9589): p. 797-9]. Herat failure is included among these pathologies. Based on this hypothesis and results obtained with medicines with anti-inflammatory properties, the authors suggest that control of this chronic inflammatory component could contribute to improvement of pathologies such as metabolic syndrome, COPD (chronic obstructive pulmonary disease), and heart failure as well as common co-morbidities in the affected patients.

In order to validate this hypothesis, it is first necessary to demonstrate its existence, which is based for the time being essentially on the increase in CRP (C-reactive protein). Taking into account the number of known factors having direct or indirect pro- or anti-inflammatory effects, and variations in expression of their soluble cellular receptors, a large-scale, detailed study of the latter seems difficult to carry out, and it seems impossible to draw conclusions from it regarding the overall result of their interactions. Another approach would therefore consist of investigating markers of points of convergence of the pathways activated or inhibited by all of these factors in order to obtain a precise indication of the presence or absence of an inflammatory state. The increase in concentration of a certain number of plasma proteins such as CRP (C-reactive protein), orosomucoid and fibrinogen reflect the existence of an inflammatory state but these raised levels are generally transient even when the inflammation persists. Only CRP can remain detectable by means of ultra-sensitive assay techniques.

The triggering of an inflammatory response by nonspecific agents such as lipopolysaccharides causes oxidative stress at the cellular level. This oxidative stress corresponds to the formation of “free radicals” of oxygen and of nitrogen which induce functional changes, reversible or not depending on their nature, of the macromolecules. It is these changes which, especially if they are irreversible, may lead to changes in metabolism, or even cell death and hence tissue damage. As noted above, many of these changes have been identified and their consequences are now often known. It is therefore important to be able on the one hand to identify the nature of the free radicals generated and especially to determine whether the organism has been able to deal with them by means of its natural defences.

As mentioned previously, the production of peroxynitrite is located at the crossroads since it is the last stage permitting “trapping” of the superoxide ions and it is the last free radical giving rise to reversible post-translational modifications (nitration of tyrosines). It can be imagined that this stage of nitration of proteins, which is reversible, constitutes a “buffer” admittedly leading to temporary adaptations or disturbances, but also enabling the chromatin DNA to be protected against oxidation and nitration, which would induce apoptosis.

As peroxynitrite and its precursors are highly diffusible, it seems logical to think that once the mechanisms of trapping and degradation of the latter are saturated, there will be extravasation to the extracellular matrix and accordingly nitration of proteins of this matrix, and therefore inter alia in the plasma. This situation would correspond to a “decompensated” nitrating stress.

This hypothesis is reinforced by our first clinical studies of peripartal asphyxia and neonatal hypoglycaemia, which have moreover been shown by others to lead to considerable nitration of the cellular proteins, particularly in the brain [Groenendaal, F., et al., Nitrotyrosine in brain tissue of neonates after perinatal asphyxia. Arch Dis Child Fetal Neonatal Ed, 2006. 91(6): p. F429-33; Groenendaal, F., et al., Nitrotyrosine in Human Neonatal Spinal Cord after Perinatal Asphyxia. Neonatology, 2007. 93(1): p. 1-6; Suh, S. W., et al., Hypoglycemic neuronal death and cognitive impairment are prevented by poly(ADP-ribose) polymerase inhibitors administered after hypoglycaemia. J Neurosci, 2003. 23(33): p. 10681-90].

It therefore certainly seems that this “decompensated” cellular nitration is accompanied by an increase in nitration of the plasma albumin which constitutes a marker of saturation of the capacities for absorption of superoxide ions and of peroxynitrite by the cell. As an association between intense nitration and the triggering of apoptosis has been described by several authors [Ischiropoulos, H. and J. S. Beckman, Oxidative stress and nitration in neurodegeneration: cause, effect, or association? J Clin Invest, 2003. 111(2): p. 163-9; Carreras, M. C. and J. J. Poderoso, Mitochondrial nitric oxide in the signaling of cell integrated responses. Am J Physiol Cell Physiol, 2007. 292(5): p. C1569-80], it seems justifiable to investigate this parameter in any pathology that is accompanied on the one hand by increased oxidative stress and on the other hand by tissue remodelling linked to an increase in apoptosis.

### Objectives

### Main Objective:

To evaluate the prognostic benefit of determining plasma nitrated albumin by ELISA in heart failure by means of a combined criterion comprising mortality and transition to a higher NYHA class.

### Main Assessment Criterion:

This is a combined criterion comprising mortality and transition to a higher NYHA class at 2 years.

This parameter is evaluated as a function of the demographic data (age, sex), past and present addictions (tobacco, alcohol), anthropometric data (height, weight, body mass index, waist/hips ratio, or even body composition), NYHA classification and the classical clinical parameters of heart failure (left ventricular ejection fraction, mean arterial pressure, heart rate, atrial fibrillation, ischaemic cardiomyopathy, data from cardio-respiratory exercise testing such as VO₂ and the respiratory equivalents of CO₂ and O₂ if available in the 6 months preceding inclusion, the aetiology and type of heart failure (systolic or diastolic), any co-morbidities (diabetes, COPD, chronic inflammatory diseases), treatments—preexisting or initiated (diuretics, ACE inhibitors, angiotensin antagonists, β-blockers, statins, aspirin, implantable defibrillators, exercise rehabilitation), and their duration as well as of the progression of HF in the monitored subjects. The phenotyping and monitoring of the RESIC 38 cohort (about 450 patients included) serve as reference.

Recruitment of the patients from the RESIC 38 cohort is completed by the day hospital in order to have patients with different NYHA stages. The controls are recruited by the CIC of Grenoble.

Evaluation is comparative relating to a series of known prognostic markers, which to some extent have been validated in HF (troponin T, NT-pro-BNP, haematocrit, adiponectin), oxidative stress (8-isoprostaglandin F2α, GSH/GSSG, vitamin E, and carbonylated albumin) and of inflammation (ultrasensitive CRP, IL-6, soluble receptor of IL-6 and IL-10) [White, M., et al., Increased systemic inflammation and oxidative stress in patients with worsening congestive heart failure: improvement after short-term inotropic support. Clin Sci (Loud), 2006. 110(4): p. 483-9; Polidori, M. C., et al., Increased F2 isoprostane plasma levels in patients with congestive heart failure are correlated with antioxidant status and disease severity. J Card Fail, 2004. 10(4): p. 334-8; Adamopoulos, S., J. T. Parissis, and D. T. Kremastinos, A glossary of circulating cytokines in chronic heart failure. Eur J Heart Fail, 2001. 3(5): p. 517-26; Anand, I. S., et al., C-reactive protein in heart failure: prognostic value and the effect of valsartan. Circulation, 2005. 112(10): p. 1428-34; de Denus, S., C. Pharand, and D. R. Williamson, Brain natriuretic peptide in the management of heart failure: the versatile neurohormone. Chest, 2004. 125(2): p. 652-68; Kistorp, C., et al., N-terminal pro-brain natriuretic peptide, C-reactive protein, and urinary albumin levels as predictors of mortality and cardiovascular events in older adults. Jama, 2005. 293(13): p. 1609-16; Moskowitz, R. and M Kukin, Oxidative stress and congestive heart failure. Congest Heart Fail, 1999. 5(4): p. 153-163; Mariani, E., et al., Oxidative stress in brain aging, neurodegenerative and vascular diseases: an overview. J Chromatogr B Analyt Technol Biomed Life Sci, 2005. 827(1): p. 65-75; Hall, C., Essential biochemistry and physiology of (NT-pro)BNP. Eur J Heart Fail, 2004. 6(3): p. 257-60; Antman, E. M., Decision making with cardiac troponin tests. N Engl J Med, 2002. 346(26): p. 2079-82; Kistorp, C., et al., Plasma adiponectin, body mass index, and mortality in patients with chronic heart failure. Circulation, 2005. 112(12): p. 1756-62].

### Objective 2:

Evaluation of the predictive value of plasma nitrated albumin for the occurrence of co-morbidities.

### Assessment Criteria:

Correlation between the concentration of plasma nitrated albumin determined at inclusion and at 1 year and the occurrence of associated pathologies (metabolic syndrome, COPD, diabetes, atherosclerosis etc.).

### Objective 3:

Evaluation of the value and if necessary of the sensitivity of plasma nitrated albumin as a marker of a chronic inflammatory condition.

### Assessment Criteria:

Correlation between the concentration of plasma nitrated albumin and recognized inflammatory parameters: ultra-sensitive CRP, IL-6 and its soluble receptor, IL-10.

### Objective 4:

Evaluation of the value and if necessary of the sensitivity of plasma nitrated albumin as a marker of “intermediate” oxidative stress.

### Assessment Criteria:

Correlation between the concentration of plasma nitrated albumin and parameters of oxidative stress corresponding to different stages of the latter:
GSH/GSSG: oxidation of thiols: “early” stage
8-isoprostaglandin F_{2α} and carbonylated albumin: peroxidation of lipids and oxidation of proteins: “advanced” stage
vitamin E: antioxidants

### Study Procedures Carried Out and Differences Relative to the Usual Management

Apart from the taking of blood samples on inclusion and at 1 year, there are no additional procedures relating to the usual patient management.

### Characteristics of the Subjects

### Recruitment

The patients are recruited during special cardiology consultations. They are then asked to take part in the study.

Recruitment of the patients from the RESIC 38 cohort is completed by the day hospital in order to have patients with different stages of the NYHA classification. The controls are recruited by the CIC of Grenoble.

Subjects meeting each of the following criteria are proposed for the study:
Age between 18 and 90 years
Person affiliated to social security or beneficiary of such a scheme
Patients with heart failure of NYHA class II to IV

Subjects meeting at least one of the following criteria cannot be included:
Pathology not connected with heart failure (e.g.: advanced neuromuscular disease, metastatic prostate cancer) and reaching life expectancy in 6 months.
A woman who is pregnant or in labour; a mother who is breast-feeding
A person deprived of freedom by a judicial or administrative decision, a person who is the subject of legal protection
A person incapable of answering the questions

Variables Measured and Methods of Measurement
Age, sex
Past and present addictions (tobacco, alcohol)
Aetiology of the cardiopathy
NYHA class
Medical history, co-morbidities
Current treatments
Arterial pressure, pulse
6 min walking distance
Body mass index
Waist, hips and pelvis measurement
Hospitalizations in connection with heart failure

Para-Clinical Parameters

The following examinations must date from less than three months relative to the date set for the various visits.
Cardiac and carotid echo-Doppler
ECG
Spirometry (FEV₁, VC) and blood gases in ambient air
Dexascann for measuring body composition

Biological Parameters
“Recognized” prognostic parameters:
   NT-pro-BNP: immunochemistry
   Troponin T: immunochemistry
   Haematocrit: optical
   Adiponectin: ELISA
Nitrating stress:
   Nitrated plasma albumin: ELISA
   Plasma albumin: immunochemistry
Inflammation:
   Ultrasensitive CRP: immunochemistry
   IL-6 and IL-6sr: ELISA
   IL-10: ELISA
Oxidative stress:
   urinary 8-isoprostaglandin F2: ELISA
   GSH/GSSG: enzymatic
   Carbonylated albumin: ELISA (under development)
   Vitamin E: HPLC
Nutritional state:
   Total plasma proteins: colorimetry
   Plasma albumin: immunochemistry
   Prealbumin (TTY): immunochemistry
Serum collection: surpluses of serum and plasma aliquoted and stored at −80° C.
Urine collection: 50 ml of urine will be aliquoted and stored at −80° C.

Statistical Analysis of the Measured Parameters

Calculation of the Number of Subjects

Calculation of the number of subjects is based on non-parametric comparison of the mortality between two groups; it is obtained from the formula following:

$$n = \frac{nA [ 1 - SA ( t ) ] + nB [ 1 - SB ( t ) ]}{2 - SA ( t ) - SB ( t )}$$

(Source: “Analyse statistique des données de survie” Catherine Hill, et al.—Médecine-sciences Flammarion)

“Survival without aggravation” is defined by the absence of event (death, transition to a higher NYHA class at 2 years).

Assuming an expected improvement in the main assessment criterion (“survival without aggravation”) of 25% between the two groups, the number of subjects to be included is 48 per group, i.e. 192 subjects for the 4 quartiles, with an alpha risk of 5% and a power of 80%, giving an odds-ratio of 1.7 between quartiles.

Strategy for Data Analysis

### Statistical Threshold and Application Conditions:

For a test, the statistical threshold (α) adopted for regarding a difference as statistically significant will be p less than or equal to 0.05.

However, to counteract the loss of power that that is introduced by multiple comparisons on non-independent parameters, the Bonferroni correction is used, with α′=α/k as statistical threshold.

The Shapiro-Wilks test is used for demonstrating the normality of the parameters.

The Levene test is used for demonstrating the homogeneity of the variances.

When the conditions for application of the parametric tests are not satisfied, non-parametric tests can be carried out.

In order to demonstrate links between quantitative parameters, the Pearson correlation test is used after verifying normality of the parameters.

In order to demonstrate links between qualitative parameters the chi-squared test is used.

### Quantitative Variables

The quantitative parameters for which normality has been assumed are described by the mean±standard deviation, the 95% confidence interval as well as the 5th and 95th percentiles.

They are expressed as median, minimum, maximum and 5th and 95th percentiles when normality is rejected.

### Qualitative Variables

The qualitative parameters are expressed as size and percentage.

The categorical variables are summarized by descriptive statistics at each time of evaluation and in each group: Size and frequency.

### Populations Analysed

An overall statistical analysis is performed on the whole population.

### Analysis of the Main Criterion

Objective 1:

To evaluate the prognostic value of determination of plasma nitrated albumin by ELISA in heart failure by means of a combined criterion comprising mortality and transition to a higher NYHA class.

Parameters:

The censoring criterion adopted is the following combined criterion: death, transition to a higher NYHA class at 2 years.

The assessment criterion is evaluated during the patient follow-up visits within each centre. Confirmation of mortality is provided at the end of the study by sending a letter to the registers of births, marriages and deaths of all the patients.

Although it is a continuous quantitative parameter, the survival time is described by its median and its 95% confidence interval as well as the quartiles, in order to take account of its asymmetric distribution.

Testing:

1.1) The benefit of the parameter (plasma nitrated albumin) is tested using the univariate Cox model, after verifying the application conditions.
1.2) Secondly, the model is adjusted for the demographic factors (age, sex, BMI) and for the recognized prognostic factors of heart failure; i.e.: the NYHA functional class, the score in the walking test, the level of ejection fraction (+ or − from 45%), the level of Plasma NT-pro BNP.
1.3) The Kaplan-Meier method is used for estimating the survival curves of each quartile for the significant factors in the adjusted Cox model.

The survival curves are compared using the Log-Rank test adjusted for all the strata (known prognostic factors).

### Results

The purpose of this study was to test the hypothesis of an increase in the production of peroxynitrite in asphyxiated children and to check whether there is a correlation between the nitrated albumin concentration and the clinical status of these children.

For this, nitrated albumin was determined in 114 plasma samples collected in the first hours of life (FHOL), at day 1 (D1) and at day 4 (D4) in 48 full-term babies who suffered perinatal asphyxia. Determination of nitrated albumin was correlated with the state of development of neonatal pathologies: neurological (encephalopathy) and systemic (coagulopathy and kidney, liver and heart damage).

17 patients developed a moderate to severe encephalopathy. The main characteristics of the patients are presented in Table I below.

TABLE I
Clinical and biochemical characteristics
of the patients investigated.

| Value | Number |
|---|---|
| Variables in the first hours | |
| pH of the umbilical artery | 7.08 ± 0.16 |
| Baseline deficit of the umbilical artery | 10.1 ± 6.6 |
| Apgar 5 minutes score | 3.0 ± 1.9 |
| IPPV at birth (n) | 41 |
| First arterial pH | 7.22 ± 0.16 |
| First baseline arterial deficit (mmol/l) | 13.3 ± 4.3 |
| First arterial lactate (mmol/l) | 11.4 ± 4.9 |
| Clinical measurements | |
| Encephalopathies (n) | 27 |
| Normal (no encephalopathy) | 21 |
| mild | 10 |
| moderate | 10 |
| severe | 7 |
| Heart damage (n) | 12 |
| Kidney damage (n) | 8 |
| Liver damage (n) | 11 |
| Coagulopathy (n) | 11 |

The variables are expressed as mean ± standard deviation.
IPPV: intermittent positive pressure ventilation.

### Correlations Between the Plasma Nitrated Albumin Concentration and the Continuous Variables

Nitrated albumin FHOL is only correlated with creatinine at D1 (r=0.38, p<0.05). Nitrated albumin at D1 is inversely correlated with the Apgar scores (r=−0.34, −0.47 and −0.33 for the Apgar scores at 1, 5 and 10 minutes, respectively, p<0.05) and with the pH FHOL (r=−0.41, p=0.01), and directly correlated with lactacidaemia FHOL (r=0.47, p<0.01, FIGS. 14A and 14B). No significant correlation was found between nitrated albumin concentrations and albumin concentrations.

The nitrated albumin concentrations corresponding to the clinical data are presented in Table I. The median of nitrated albumin concentration (25-75th percentiles) at D1 increases with the severity of the neonatal encephalopathy (NE): 7.0 (5.3-8.6) ng/ml in children not developing NE, 8.3 (4.7-10.4) ng/ml in children with a mild NE, 13.1 (8.4-20.3) ng/ml in the case of mild NE and 16.7 (13.1-24.8) ng/ml in the case of a severe NE (χ²=7.23, p<0.05, FIGS. 15A-C). The differences in nitrated albumin concentrations between the 4 subgroups of NE reach a significativity level only when the Bonferroni correction is not applied to the differences in nitrated albumin at D1 between moderate NE and no NE (z-score: 2.2) and between severe NE and no NE (z-score: 1.98). Plasma nitrated albumin at D1 was significantly higher in the neonates with a moderate form of NE than in neonates with a mild form of NE or without NE (median and 25-75th percentiles: 14.4 and 8.4-23.7 ng/ml versus 7.3 and 4.4-9.2 ng/ml, respectively, χ²=6.14, p=0.01, FIG. 16).

The levels of nitrated albumin at day 1 are increased in patients who develop a moderate or severe form of NE compared with that of patients with a normal neurological profile or who develop a mild NE (median: 14.4 ng/ml versus 7.3 ng/ml, respectively, p=0.01).

In contrast, the nitrated albumin concentration at D1 is not associated with systemic complications. The nitrated albumin concentrations in the first hours of life and at D4 do not differ with respect to the neonatal neurological data.

### Conclusion:

The results indicate that significant nitrating stress occurs in the course of severe perinatal asphyxia.

Surprisingly, plasma nitrated albumin appears to be a good marker of the neurological development of full-term babies presenting perinatal asphyxia.

### Example 6

### Obtaining, Screening and Characterization of the Hybridomas

### 1—Immunization

5 female OF1 Charles River mice (18-20g) were immunized by intravenous and subcutaneous injection of a mixture of peptides SEQ ID NO 16 and SEQ ID NO 17, the two peptides being coupled to ovalbumin in the presence of Freund's complete adjuvant. 3 mice (mice 1 to 3) received 50 μg of the mixture of peptides and 2 mice (mice 4 and 5) received 10 μg of the mixture of peptides.

3 weeks after the first injection, the mice were reinjected with the mixture of two peptides in the presence of Freund's incomplete adjuvant (1st booster).

3 weeks after the second injection, the mice were reinjected with the mixture of two peptides in the presence of Freund's incomplete adjuvant (second booster).

1 month after the first injection, serum samples were taken from the injected mice, and said sera were tested for the presence of antibodies directed against nitrated albumin.

Only the sera from mice 2 and 5 had antibodies that recognize nitrated albumin, and were therefore kept.

10 days after the serum test, mouse 2 received an intraperitoneal and intravenous booster of 20 μg of the mixture of peptides. The spleens were removed 3 days after the booster.

1 month after the serum test, mouse 5 received an intraperitoneal and intravenous booster of 10 μg of the mixture of peptides. The spleens were removed 3 days after the booster.

### 2—Cellular Fusion

Mice 2 and 5 were bled and their spleen was removed under sterile conditions with DMEM (Dulbecco's Modified Eagle's Medium). The spleens were ground and filtered with a strainer. In parallel, the intraperitoneal cavity of the mice was washed with DMEM, and the DMEM containing macrophages was recovered. The macrophages were counted in a Malassez cell in order to prepare a solution at 104 macrophages/ml in DMEM HAT SVF 20% ATB medium (DMEM, 4 mM glutamine, HAT (hypoxanthine 100 μM, aminopterin 0.4 μM, thymidine 16 μM), 20% decomplemented fetal calf serum, 1% antibiotics (penicillin/streptomycin)).

The splenocytes obtained were then washed three times with DMEM.

In parallel, myeloma cells from BalB/c Sp2/O Ag14 mice (ATCC No. CRL 1581) were also washed 3 times in DMEM.

The splenocytes and the myeloma cells were mixed at a splenocytes/myeloma ratio of 5/1 and centrifuged at 244 g for 7 minutes.

The supernatant was removed and 1 ml of PEG (40% solution of polyethylene glycol, MW 1500 heated to 37° C.) was added.

The cells were centrifuged at 800 rpm (108 g) for 12 minutes, 10 ml of DMEM HAT SVF 20% medium (DMEM, 4 mM glutamine, HAT (hypoxanthine 100 μM, aminopterin 0.4 μM, thymidine 16 μM), 20% decomplemented fetal calf serum) was added slowly.

The cells were centrifuged at 1200 rpm (244 g) for 7 minutes, the supernatant was removed and a volume v of DMEM HAT SVF 20% medium was added to the pellet such that: v (in ml)=number of splenocytes/107 (i.e. 107 cells/ml)

The tube was left at ambient temperature for 1 hour before being returned carefully for resuspending the cells.

In 96-well plates, 100 μl/well of the solution at 104 macrophages/ml was added and then 100 μl per well of fused cells was added at the following dilutions:

dilution 1/10: 3 plates at 105 splenocytes per well
dilution 1/20: 5 plates (2×50 ml) at 5×104 splenocytes per well
dilution 1/40: 2 plates at 2.5×104 splenocytes per well

The plates were put in an oven at 37° C., 5% CO₂ for 10 days.

### 3—Selection of the Hybridomas

After 10 days of culture of the fusion products, two selection tests were carried out:
Test 1: The wells in which the cells have reached confluence were analysed:
   for the fusions obtained from the splenocytes of mouse 2, 164 wells were analysed,
   for the fusions obtained from the splenocytes of mouse 5, 407 wells were analysed.

100 μl of supernatant was taken from each of these wells and the supernatants were tested by ELISA for detecting the required antibody (cf. Screening of the supernatants from the anti-nitrated albumin hybridomas).

After the ELISA assay, the selected cells (secreting antibodies directed against nitrated albumin) were passaged in 0.4 ml of medium in wells of a 24-well plate.

When the cells began to multiply (24 to 48 hours), 1 mL of DMEM HAT SVF 15% HCF 1% ATB medium (DMEM, 4 mM glutamine, HAT (hypoxanthine 100 μM, aminopterin 0.4 μM, thymidine 16 μM), 15% decomplemented fetal calf serum, 1% HCF (hybridoma cloning factor macrophage-like origin), 1% antibiotics (penicillin/streptomycin)) was added.
Test 2: The wells in which the cells had reached confluence were analysed:
   for the fusions obtained from the splenocytes of mouse 2, 1 well of the 24-well plate was analysed,
   for the fusions obtained from the splenocytes of mouse 5, 6 wells of the 24-well plate were analysed.

100 μl of supernatant was taken from each of these wells and the supernatants were tested by ELISA for detecting the required antibody (cf. Screening the supernatants from the anti-nitrated albumin hybridomas).

4—Screening the Supernatants from the Anti-Nitrated Albumin Hybridomas

Antigens (Ag) used: Nitrated albumin, albumin, KLH-peptide SEQ ID NO 16 or KLH-peptide SEQ ID NO 17.

| STAGES | CONDITIONS | |
|---|---|---|
| Coating | | |
| (adsorption of the Ag) | 96-well plate (Maxisorp, Nunc) | |
| Concentration of the Ag | 1 | μg/ml |
| Buffer | PBS | |
| Volume / well | 50 | μl |
| Incubation | overnight at ambient temperature | |
| Washing: ×1 | PBS - 0.05% (v/v) Tween 20 | |
| Saturation | | |
| Buffer | PBS-milk 2.5% (w/v) | |
| Volume / well | 150 | μl |
| Incubation | 1 h at 25° C. | |
| Washing: ×1 | PBS - 0.05% (v/v) Tween 20 | |
| Antibody to be tested | | |
| Supernatant from pure | | |
| culture | | |
| Volume/well | 50 | μl |
| Incubation | 2 h at 25° C. | |
| Washing: ×3 | PBS - 0.05% (v/v) Tween 20 | |
| Secondary antibody | | |
| (conjugated peroxidase) | Anti-IgG and IgM (115-036-044, Jackson) | |
| Dilution | 1/10 000 | |
| Buffer | PBS-0.05% (v/v) Tween 20-0.5% (w/v) BSA | |
| Volume/well | 50 | μl |
| Incubation | 1 h at 25° C. | |
| Washing: ×3 | PBS - 0.05% (v/v) Tween 20 | |
| Detection | | |
| Reagent | Tetramethylbenzidine (50-76-05, KPL, Inc.) | |
| Volume/well | 50 | μl |
| Incubation | 10 | min |
| Stopping the reaction | H₂SO₄ 1M (S1526, Sigma) | |
| Volume | 50 | μl |

### 5—Isotyping of the Antibodies

The isotype of the antibodies was determined using the kit SouthernBiotech SBA Clonotyping System/HRP (Cliniscience, Montrouge, France) as follows:
1. Coating the Plates
Dilute the mouse anti-immunoglobulin antibody to a concentration of 5 μg/ml. Deposit 50 μl per well and incubate for 1 hour at 37° C. or 16 hours at ambient temperature.
2. Washing
Rinse once with 200 μl/well of PBS-Tween 20 0.05%% (v/v).
3. Blocking
Add to each well 150 μl of PBS-Milk 2.5% (w/v) and incubate for 1 hour at 37° C.
4. Washing
Rinse once with buffer PBS-Tween20 0.05% (v/v).
5. Preparation of the samples of antibody to be tested
Dilute the hybridoma culture supernatants to 1/10 in PBS-Tween20 0.05% (v/v)-BSA 0.5% (w/v). Deposit 50 μl per well and incubate for 2 hours at ambient temperature.
6. Washing
Rinse 3 times with buffer PBS-Tween20 0.05% (v/v).
7. Secondary antibody
Deposit 50 μl per well of anti-mouse IgA, IgG1, IgG2a, IgG2b, IgG3 or IgM antibodies conjugated with peroxidase (HRP) (diluted to 1/2000 in PBS-Tween20 0.05% (v/v)-BSA 0.5% (w/v)) and incubate for 1 hour at ambient temperature.
8. Washing
Rinse 3 times with buffer PBS-Tween20 0.05% (v/v).
9. Reaction with the substrate
Deposit 50 μl per well of tetramethylbenzidine (KPL, Inc.) and incubate the plate for 10 minutes at ambient temperature.
10. Stopping the reaction
Add 50 μl of H₂SO₄ to each well and read the absorbance at 450 nm, on the microplate reader (Dynex).

### 6—Results

7 hybridomas were stored: 2F3, 11G6, 12F5, 12H3, 13H8, 13H10 and 15F8 on account of their excellent affinity for the peptides corresponding to the nitrated human albumin sequences (SEQ ID NO 16 and SEQ ID NO 17) and Alb-NO₂, as shown in Table II below.

TABLE II
Optical density and isotype
of the 7 clones selected.

| | OD on | OD on | OD on | |
|---|---|---|---|---|
| | Alb-NO2 | SEQ ID NO 17 | SEQ ID NO 16 | Isotype |
| 2F3 | 1.172 | 2.211 | 0.275 | IgG1 |
| 11G6 | 1.355 | 0.081 | 0.081 | IgM |
| 12F5 | 0.061 | 0.232 | 0.052 | IgM, IgG1, |
| | | | | IgG2b |
| 12H3 | 0.066 | 1.006 | 0.056 | IgG1 |
| 13H8 | 0.104 | 0.063 | 1.837 | IgG1, IgM |
| 13H10 | 1.449 | 0.059 | 2.562 | IgG2b |
| 15F8 | 1.461 | 0.064 | 2.225 | IgA, IgG2b |

Tests of coating specificity were also conducted on nitrated KLH albumin, nitrated insulin and nitrated haemoglobin. All the antibodies are negative on albumin, nitrated KLH, nitrated insulin and nitrated haemoglobin.

Clones 2F3 and 13H10 were stored and cloned, carrying out limiting dilutions in 96-well plates containing 10, 5, 3, 1 or 5 cells per well on average.

The cloning products were frozen in Medium DMEM HT SVF 15% HCF 1% ATB medium (2): DMEM, 4 mM glutamine, HAT (hypoxanthine 100 μM, thymidine 16 μM), 15% decomplemented fetal calf serum, 1% HEF (hybridoma enhancing supplement), 1% antibiotics (penicillin/streptomycin) made up with 10% DMSO (dimethylsulphoxide).

### Example 7

### Validation of the Use of mAB Anti-alb-NO₂-Tyr¹³⁸ (Clone 13H10) as Capture Antibody in ELISA for Assaying Nitrated Albumin

In this form of ELISA, the monoclonal antibody anti-alb-NO₂-Tyr138 (clone 13H10) is used for capture and an anti-albumin polyclonal antibody conjugated with peroxidase is used for detection. The plates are revealed with TMB (3,3′,5,5′-tetramethylbenzidine), a chromogenic substrate of peroxidase the colour of which becomes yellow in the presence of sulphuric acid (stopping the reaction). The reaction can be quantified by detection at 450 nm.

The results obtained show that the anti-alb-NO₂-Tyr138 monoclonal antibody can be used as capture antibody in sandwich ELISA (FIG. 7).

The dose-response curve shows a limit of detection of nitrated albumin of 1 ng/ml.

### Example 8

### Effect of the Serum on ELISA of Nitrated Albumin Using mAB Anti-alb-NO₂-Tyr¹³⁸ (Clone 13H10) for Capture (FIG. 8)

In this form of ELISA, the anti-alb-NO₂-Tyr¹³⁸ monoclonal antibody is used for capture and an anti-albumin polyclonal antibody conjugated with peroxidase is used for detection. The plates are revealed with TMB.

A mixture (pool) of native human serum at different dilutions is added to the range of nitrated albumin. The curves show that the serum diluted to 1:20 only interferes significantly with the determination of nitrated albumin for concentrations of the latter <10 ng/ml. At greater dilutions, the serum does not display significant interference. It should be noted that this serum mixture also naturally contains “endogenous” nitrated albumin.

### Example 9

### Effect of the Reduced Serum on ELISA of Nitrated Albumin Using mAB Anti-alb-NO₂-Tyr¹³⁸ (Clone 13H10) for Capture (FIGS. 9 and 10)

In this form of ELISA, the anti-alb-NO₂-Tyr138 monoclonal antibody is used for capture and an anti-albumin polyclonal antibody conjugated with peroxidase is used for detection. The plates are revealed with TMB.

In order to avoid interference from endogenous nitrated albumin, the serum was reduced using sodium dithionite (0.1 M, pH 9.0, 1 h). This treatment reduces the nitrotyrosine residues to aminotyrosines, which are not recognized by the antibody.

### Example 10

### Effect of the Reduced Albumin on ELISA of Nitrated Albumin Using mAB Anti-alb-NO₂-Tyr¹³⁸ (Clone 13H10) for Capture (FIG. 11)

In this form of ELISA, the anti-alb-NO₂-Tyr138 monoclonal antibody is used for capture and an anti-albumin polyclonal antibody conjugated with peroxidase is used for detection. The plates are revealed with TMB.

Human serum albumin reduced with sodium hydrosulphite (0.1 M, pH 9.0, 1 h) is added at different concentrations to the range of nitrated albumin.

It is found that the reduced albumin only interferes at concentrations ≧150,000 times those of nitrated albumin.

### Example 11

### Validation of the Use of mAB Anti-alb-NO₂-Tyr138 (Clone 13H10) as Detecting Antibody in ELISA for Determining Plasma Nitrated Albumin (FIG. 12)

In this form of ELISA, an anti-albumin polyclonal antibody is used for capture and the anti-alb-NO₂-Tyr138 monoclonal antibody conjugated with peroxidase is used for detection. The plates are revealed with TMB.

The results obtained show that the anti-alb-NO₂-Tyr138 monoclonal antibody can be used as detecting antibody in sandwich ELISA.

### Example 12

### Validation of the Use of mAB Anti-alb-NO₂-Tyr138 (Clone 13H10) in ELISA for Determining Nitrated Albumin in Human Sera (FIG. 13)

In this form of ELISA, the anti-alb-NO₂-Tyr138 monoclonal antibody is used for capture and an anti-albumin polyclonal antibody conjugated with peroxidase is used for detection. The plates are revealed with TMB.

A mixture of human sera is assayed at different dilutions in PBS. The results show that nitrated albumin can be assayed in the serum up to a dilution of the latter at 1:50.

### Example 13

### Antibody Directed Against the Sequence LVRY—NO₂TQKAPQ which Includes Nitrotyrosine in Position 411 of the Albumin

Results similar to those described above were obtained with the 2F3 monoclonal antibody directed against the sequence LVR(Y—NO₂)TQKAPQ which includes nitrotyrosine in position 411.

In other words, this antibody shows the same specificity and affinity with respect to nitrated albumin and functions as a capture or detection antibody.

### CONCLUSIONS

The results obtained show that the monoclonal antibodies directed against the known nitrated sequences of human serum albumin comprising respectively Tyr¹³⁸ and Tyr⁴¹¹:
specifically recognize these two sequences within native nitrated human albumin
do not recognize non-nitrated human albumin
do not recognize other nitrated peptides or proteins such as insulin, haemoglobin and KLH
can be used for determining nitrated human albumin by ELISA
can be used for determining nitrated human serum albumin by ELISA in human serum
can be used for capture or for detection in ELISA

1-27. (canceled)

28. Method of in vitro diagnosis of the state of severity and progressiveness of a chronic pathology associated with nitrating stress in a biological sample from an individual, comprising:
quantitative assay, in a biological sample from an individual, of the degree of nitration of a first particular nitrated protein or physiological peptide sequence,
quantification of said degree of nitration of said first particular protein or physiological peptide sequence by comparing the concentration of said first particular nitrated protein or physiological peptide sequence with the total concentration of said first particular protein or physiological peptide sequence, nitrated and non-nitrated, obtained from a biological sample from the same individual,
comparing said degree of nitration of said first particular nitrated protein or physiological peptide sequence with the degree of nitration of a second particular protein or physiological peptide sequence obtained from a biological sample from an individual not affected by said chronic or acute pathology,
said second particular protein or physiological peptide sequence being a variant or an isoform, or having an amino acid sequence identity of at least 90%, and in particular 100%, with the aforesaid first particular protein or physiological peptide sequence or also derived from the same wild-type or mutated gene,
said first particular protein or physiological peptide sequence being nitrated on at least one tyrosine residue located in an equivalent position in the aforesaid second particular protein or physiological peptide sequence,
deduction, from the comparison carried out in the preceding stage, of the degree of nitrating stress of the individual, which may correspond to a state of severity and progressiveness of said chronic or acute pathology.

29. Method of in vitro diagnosis according to claim 28, said method comprising:
quantitative assay, in a biological sample from an individual, of the degree of nitration of a first particular nitrated physiological protein or peptide sequence,
quantification of said degree of nitration of said first particular protein or physiological peptide sequence by comparing the concentration of said first particular nitrated protein or physiological peptide sequence with the total concentration of said first particular protein or physiological peptide sequence, nitrated and non-nitrated, obtained from a biological sample from the same individual,
comparing said degree of nitration of tyrosine residues of the first particular nitrated protein or physiological peptide sequence with the degrees of nitration of a set of n particular proteins or physiological peptide sequences, the value of the degree of nitration of each of the aforesaid n particular proteins or peptide sequences being known and associated respectively with a particular state of severity and progressiveness of said chronic or acute pathology,
said n particular proteins or physiological peptide sequences being variants or isoforms of the aforesaid first particular protein or peptide sequence, or having an amino acid sequence identity of at least 90%, and in particular 100%, with the aforesaid first particular protein or physiological peptide sequence,
said n particular proteins or physiological peptide sequences being nitrated on at least one tyrosine residue located in an equivalent position to the tyrosine residue in the aforesaid first particular protein or physiological peptide sequence,
deduction, from the comparison carried out in the preceding stage, of the degree of nitrating stress of the individual which may correspond to a state of severity and progressiveness of said chronic or acute pathology.

30. Method of in vitro diagnosis according to claim 28, characterized in that in vitro quantitative assay is carried out by means of at least one antibody, each antibody specifically recognizing a nitrated tyrosine residue of a particular nitrated protein or physiological peptide sequence.

31. Method of in vitro diagnosis according to claim 28, characterized in that the particular nitrated physiological protein is preferably a circulating protein, in particular selected from the following proteins: albumin, prealbumin, vitamin D binding protein (VDBP), transferrin, ceruloplasmin, retinol binding protein (RBP), insulin, haemoglobin, β actin, band 3 protein of erythrocyte anion transporter, β chain of erythrocyte spectrin, fibronectin precursor, β chain of fibrinogen and erythrocyte membrane protein band 4.1.

32. Method of in vitro diagnosis according to claim 28, characterized in that the chronic or acute pathology associated with nitrating stress belongs to the following group: inflammatory diseases, infectious diseases, neurodegenerative diseases, hypoxic and ischaemic diseases, diabetes, metabolic disorders, cardiovascular and respiratory diseases, and cancer.

33. Method of in vitro diagnosis according to claim 28, said method comprising:
detecting an immune complex resulting from bringing at least one antibody specifically recognizing a nitrated tyrosine residue of physiological albumin into contact with nitrated physiological albumin obtained from a biological sample from an individual, said detection of the immune complex permitting the determination of the degree of nitration of said nitrated physiological albumin,
comparing said degree of nitration of tyrosine residue of physiological albumin, with the degrees of nitration of tyrosine residues of a set of n physiological albumins the value of the degree of nitration of which is known and is associated respectively with a particular state of severity and progressiveness of said chronic or acute pathology,
deducing, from the comparison carried out in the preceding stage, the degree of nitrating stress of the individual, which may correspond to a state of severity and progressiveness of said chronic or acute pathology.

34. Method of in vitro diagnosis according to claim 33, characterized in that said antibody recognizes the nitration of human albumin on the Y¹³⁸ tyrosine residue.

35. Method of in vitro diagnosis according to claim 33, characterized in that said antibody recognizes the nitration of human albumin on the Y⁴¹¹ tyrosine residue.

36. Method of in vitro diagnosis according to claim 33, said method comprising:
detection, by means of an antibody directed against albumin, of an immune complex resulting from bringing at least one specific antibody which recognizes the nitration of human albumin on the Y¹³⁸ tyrosine residue, into contact with nitrated physiological albumin obtained from a biological sample from an individual, said detection of the immune complex permitting determination of the degree of nitration of nitrated physiological albumin,
comparing said degree of nitration of tyrosine residue of nitrated physiological albumin, with the degrees of nitration of tyrosine residues of a set of n physiological albumins of which the value of the degree of nitration of tyrosine residues of nitrated physiological albumin is known and associated respectively with a particular state of severity and progressiveness of said chronic or acute pathology,
deducing, from the comparison carried out in the preceding stage, the degree of nitrating stress of the individual, which may correspond to a state of severity and progressiveness of said chronic or acute pathology.

37. Method of in vitro diagnosis according to claim 33, characterized in that the degree of nitrating stress of the individual may correspond to a state of severity and progressiveness of pathologies selected from: inflammatory diseases, infectious diseases, neurodegenerative diseases, hypoxic and ischaemic diseases, diabetes, metabolic disorders, cardiovascular and respiratory diseases, and cancer.

38. Method of in vitro diagnosis according to claim 33, characterized in that said detection of the immune complex is carried out by the techniques of immunohistochemistry, immunocytochemistry, immunoprecipitation, Western blot or radioimmunology.

39. Antibody specifically recognizing nitrated albumin on the Y¹³⁸ tyrosine residue, in particular a monoclonal antibody.

40. Monoclonal antibody according to claim 39, secreted by the hybridoma deposited at the CNCM (Collection Nationale de Culture de Microorganismes, Institut Pasteur, Paris, France) on 8 Jan. 2009, under the accession number CNCM I-4111.

41. Antibody specifically recognizing nitrated albumin on the Y⁴¹¹ tyrosine residue, in particular a monoclonal antibody.

42. Monoclonal antibody according to claim 41, secreted by the hybridoma deposited at the CNCM (Collection Nationale de Culture de Microorganismes, Institut Pasteur, Paris, France) on 8 Jan. 2009, under the accession number CNCM I-4110.

43. Hybridoma deposited at the CNCM (Collection Nationale de Culture de Microorganismes, Institut Pasteur, Paris, France) on 8 Jan. 2009, under the accession number CNCM I-4111.

44. Hybridoma deposited at the CNCM (Collection Nationale de Culture de Microorganismes, Institut Pasteur, Paris, France) on 8 Jan. 2009, under the accession number CNCM I-4110.

45. Method of in vitro preparation of a particular nitrated protein or peptide sequence on tyrosine residues in which the nitrated tyrosine residues correspond to the physiologically nitrated tyrosine residues of a particular nitrated protein or physiological peptide sequence in chronic or acute pathologies associated with nitrating stress,
said particular nitrated protein or peptide sequence having an amino acid sequence identity of at least 90%, and in particular 100%, with the aforesaid particular nitrated physiological protein, even gene comprising:
in vitro nitration of a particular protein or peptide sequence by tetranitromethane in a molar ratio to the nitratable tyrosines of the particular protein or peptide sequence not exceeding 20:1, in an aqueous buffer with pH between 7.5 and 8.5,
in vitro identification of the nitrated tyrosines of said particular nitrated protein, in particular by mass spectrometry, and
optionally, comparing the nitrated tyrosines identified on said particular protein or peptide sequence with the tyrosine residues of the particular nitrated protein or physiological peptide sequence.

46. Method of in vitro diagnosis according to claim 33, said method comprising:
detection, by means of an antibody directed against albumin, of an immune complex resulting from bringing at least one specific antibody which recognizes the nitration of human albumin on the Y⁴¹¹ tyrosine residue, into contact with nitrated physiological albumin obtained from a biological sample from an individual, said detection of the immune complex permitting determination of the degree of nitration of nitrated physiological albumin,
comparing said degree of nitration of tyrosine residue of nitrated physiological albumin, with the degrees of nitration of tyrosine residues of a set of n physiological albumins of which the value of the degree of nitration of tyrosine residues of nitrated physiological albumin is known and associated respectively with a particular state of severity and progressiveness of said chronic or acute pathology, deducing, from the comparison carried out in the preceding stage, the degree of nitrating stress of the individual, which may correspond to a state of severity and progressiveness of said chronic or acute pathology.
